(19) United States
(12) Patent Application Publication
(10) Pub. No.: US 2011/0137018 A1
(43) Pub. Date: Jun. 9, 2011

(54) MAGNETIC SEPARATION SYSTEM WITH PRE AND POST PROCESSING MODULES

(75) Inventors: David A. Chang-Yen, Oxnard, CA (US); Jafar Darabi, Goleta, CA (US); Yanting Zhang, Santa Barbara, CA (US); Paul Pagano, Moorpark, CA (US); Frederick W. Gluck, Westlake Village, CA (US)

(73) Assignee: CYNVENIO BIOSYSTEMS, INC., Westlake Village, CA (US)

(21) Appl. No.: 12/937,983

(22) PCT Filed: Apr. 16, 2009
(86) PCT No.: PCT/US09/40866

§ 371 Date: Feb. 11, 2011

Related U.S. Application Data
(62) Division of application No. 61/124,565, filed on Apr. 16, 2008.

(51) Int. Cl. C07K 1/14; C07H 21/02; B03C 1/00; B03C 1/02; B03C 1/01

(52) U.S. Cl. 530/412; 536/23.1; 209/8

(57) ABSTRACT

A system for sorting and trapping magnetic target species includes a microfluidic chamber designed to receive and then temporarily hold magnetic particles in place within the module. A pre-processing module may mix a sample and magnetic particles to cause certain species in the sample to be labeled. The micorfluidic chamber may include a mechanism to move magnetic particles within the chamber. A post-processing module or the microfluidic chamber may be used to separate the labeled species from the magnetic particles by adding a release reagent. The magnetic particles and/or their payloads may be released and separately collected at an outlet of the chamber or the post-processing module.

### CROSS-REFERENCE TO RELATED APPLICATIONS

This application claims priority under U.S.C. §119 to provisional application 61/124,565, titled “MAGNETIC CELL SORTING SYSTEM WITH MIXING MODULES,” filed on Apr. 16, 2008, the disclosure of which is incorporated herein in its entirety for all purposes.

### FIELD OF INVENTION

This invention pertains generally to magnetic separation systems. More specifically, this invention pertains to the design and mechanism of a magnetic separation system with pre and post processing modules.

### BACKGROUND

Sorting small amounts of biological and non-biological material is an important capability in biology and medicine. The target and/or non-target species may comprise small or large chemical entities of natural or synthetic origin such as chemical compounds, supermolecular assemblies, proteins, cells, viruses, bacteria, organelles, other intracellular materials, fragments, analytes, glasses, ceramics, etc. Magnetic Activated Cell Sorting (MACS) is sometimes used as a cell sorting technique because it allows the rapid selection of a large number of target cells. The applications of MACS span a broad spectrum, ranging from protein purification to cell based therapies. Typically, target materials are labeled with one or more superparamagnetic particles that are conjugated to a molecular recognition element (e.g. a monoclonal antibody) which recognizes a particular surface marker of the target.

In order to achieve high throughput and high recovery of the rare target materials (or other target components), improvements on existing MACS systems are needed.

### SUMMARY

A system for sorting and trapping magnetic target species includes a microfluidic sorting chamber designed to receive and then temporarily hold magnetic particles in place within the module. A pre-processing and/or post-processing module is in fluidic communication with the sorting chamber. A pre-processing module may mix a sample and magnetic particles to cause certain species in the sample to be labeled. The microfluidic chamber may include a mechanism to move magnetic particles within the chamber. A post-processing module or the microfluidic chamber may be used to separate the labeled species from the magnetic particles by adding a release reagent. The magnetic particles and/or their payloads may be released and separately collected at an outlet of the chamber or the post-processing module.

In various embodiments, a fluidic sorting and trapping system is designed or adapted to receive, label one or more species in a sample, and then temporarily hold magnetic particles in place within a sorting chamber or module. The captured species are then released, collected, and/or further processed. In such embodiments, the magnetic particles flowing into the sorting chamber are trapped there while the other sample components (non-magnetic) continuously flow through and out of the chamber, thereby separating and concentrating the species captured on the magnetic particles. Only after the non-magnetic sample components have flowed out of the sorting chamber are the magnetic particles and/or their payloads released and separately collected at an outlet of the sorting chamber.

According to various embodiments, magnetic particles are subjected to hydrodynamic forces within a region of fluidics system such as a chamber on a unitary fluidics device in order to facilitate labeling magnetic particles, releasing captured species from magnetic particles or otherwise processing a fluid medium containing magnetic particles. In a pre-processing module, one or more reservoirs on the fluidic device may receive a fluid medium containing a sample, magnetic particles, and/or a selection entity such as an antibody marker. These components may be delivered separately to different reservoirs, e.g., a sample reservoir and a magnetic particle reservoir. These reservoirs may be in fluid communication with each other and with the sorting chamber. Valves between reservoirs and the sorting chamber control pre-processing flow and processing flow.

In certain embodiments, contents of the reservoirs may be mixed by moving the fluid medium from one reservoir to another. For example, the fluid medium may be from different reservoirs may be mixed via pneumatic pressure, magnetic field, ultrasonic agitation, stirring, and the like. The pre-processing may incubate or label a sample with magnetic particles and selection entities. In some embodiments, pre-processing may include washing raw magnetic particles, for example, magnetic particles containing preservatives, with a wash buffer prior to labeling.

While the magnetic particles and the bound species are temporarily trapped in the sorting chamber, buffer flow may remove unlabeled and other material from the chamber. Further, the buffer flow may be stopped to allow agitation of the magnetic particles and bound species trapped in the sorting chamber. According to various embodiments, this agitation and movement may further release unlabeled and unwanted material from being physically immobilized among the magnetic particles. This agitation and movement may be caused by magnetic forces induced by alternating magnets, ultrasonic waves, mechanical stirring, pneumatic pressure, and other forces. The magnetic particles and bound species may be then immobilized again while more buffer flows through the sorting chamber to further remove the unlabeled and unwanted material.

In still other embodiments, post processing operations may be performed on the trapped and concentrated magnetic particles with bound species in the sorting chamber or in a post-processing module. Reagents may be added to lyse cells, further react with the trapped material, or release the bound species from the magnetic particles. In certain embodiments, the magnetic particles and/or the released species may be separately collected at an outlet of the chamber or the post-processing module.

These and other features and embodiments of the invention will be described in more detail below with reference to the associated drawings.

### BRIEF DESCRIPTION OF THE DRAWINGS

FIG. 1A shows a system that employs disposable fluidics chips or cartridges in accordance with various embodiments of the present invention.

FIG. 1B is a process flow diagram showing a method of using the system of FIG. 1A.

FIGS. 1C and 1D illustrates a top view and a side view of a magnetic trapping module in accordance with certain embodiments.

FIG. 2A is a flow chart depicting a sequence of operations that may be employed to label sample species using pneumatic mixing of contents in two reservoirs on a fluidics device.

FIG. 2B is a schematic diagram illustrating a fluidics device having multiple on chip reservoirs.

FIGS. 2C and 2D are cross sectional views of an on-chip reservoir design that may be employed with the fluidics device of FIG. 2B or other devices.

FIG. 2E presents various alternative embodiments for facilitating on chip labeling of sample species in accordance with various embodiments of the present invention.

FIG. 2F presents a magnetic center pole mixing system for facilitating on chip labeling of sample species.

FIG. 2G is a simplified diagram depicting the effects of oscillation of a magnetic field gradient on magnetic particles.

FIG. 2H is a flow chart depicting a sequence of operations that may be employed to release bound sample from magnetic particles, typically after sorting.

FIG. 2I is a force diagram showing magnetic forces acting on magnetic bead bound target species under the influence of a varying magnetic field.

FIG. 2J is a schematic diagram depicting an apparatus and method for trapping magnetically labeled cells, removing non-specifically bound cells, and releasing bound cells from the magnetic particles.

FIG. 3A depicts a fluidics input for a sample well and a bead release reagent well.

FIG. 3B shows a structure of a magnetic trap disposed in a fluidics device for post-capture treatment of target species.

FIG. 4A-4H depicts examples of different types of ferromagnetic MFG structures that may be employed with this invention.

FIG. 5 presents examples of non-magnetic capture features fabricated among a soft-magnetic (e.g., nickel) pattern.

FIG. 6A-6C shows examples of random array of ferromagnetic structures.

### DESCRIPTION OF CERTAIN EMBODIMENTS

Introduction and Context

Magnetic Activated Cell Sorting (MACS) systems are capable of high-purity selection of the labeled sample components. In certain embodiments these systems operate in a “trapping mode” where the non-target and target species are sequentially eluted after the application of the external magnetic field. In other words, the species attached to magnetic particles are held in place while the unattached species are eluted. Then, after this first elution step is completed, the species that are attached magnetic field and were prevented from being eluted are freed in some manner such that they can be eluted and recovered. In other embodiments, the systems operate in a “deflection mode” in which labeled and unlabeled species from a sample flow through a sorting region exposed to a magnetic field, and those species labeled with magnetic particles are deflected into an outlet chamber where they can be collected in purified form.

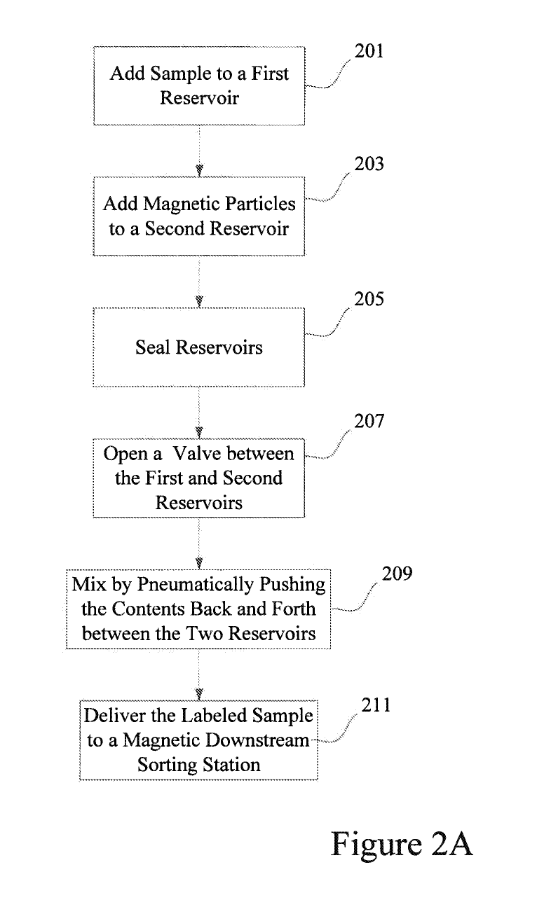

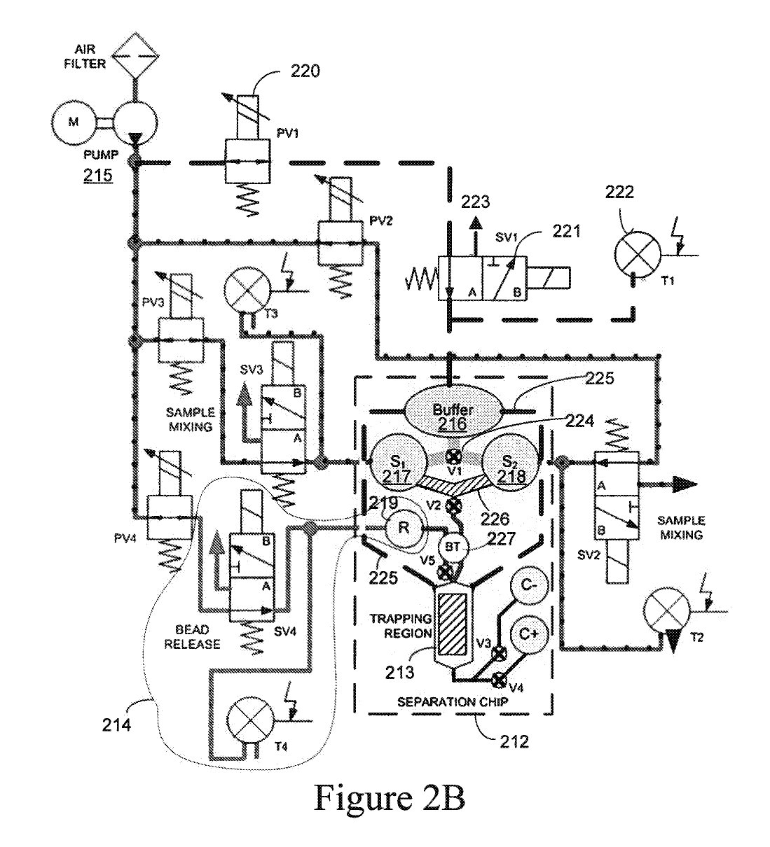

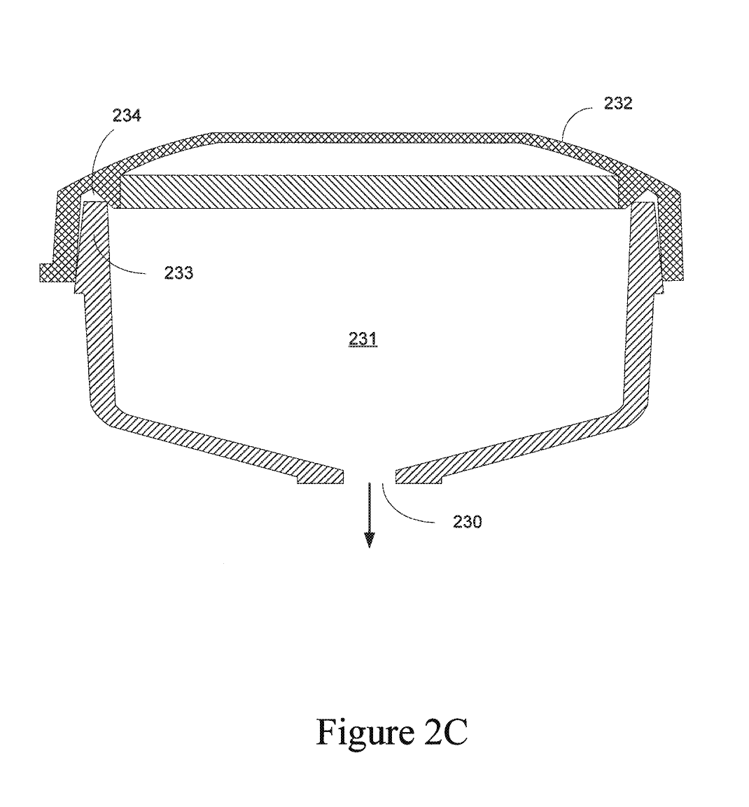

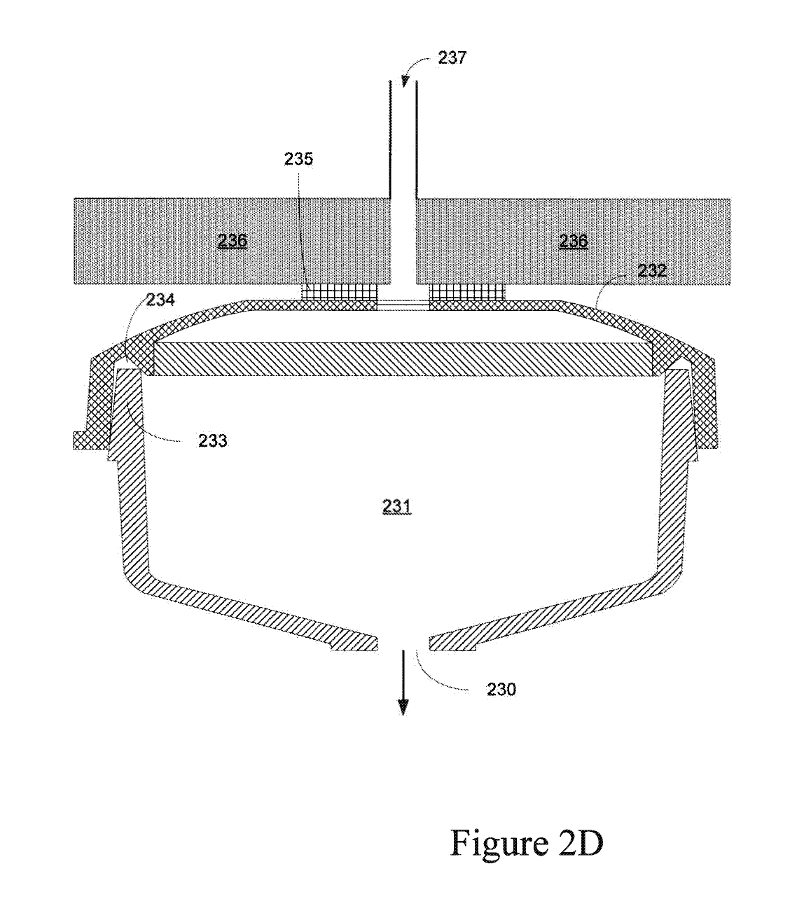

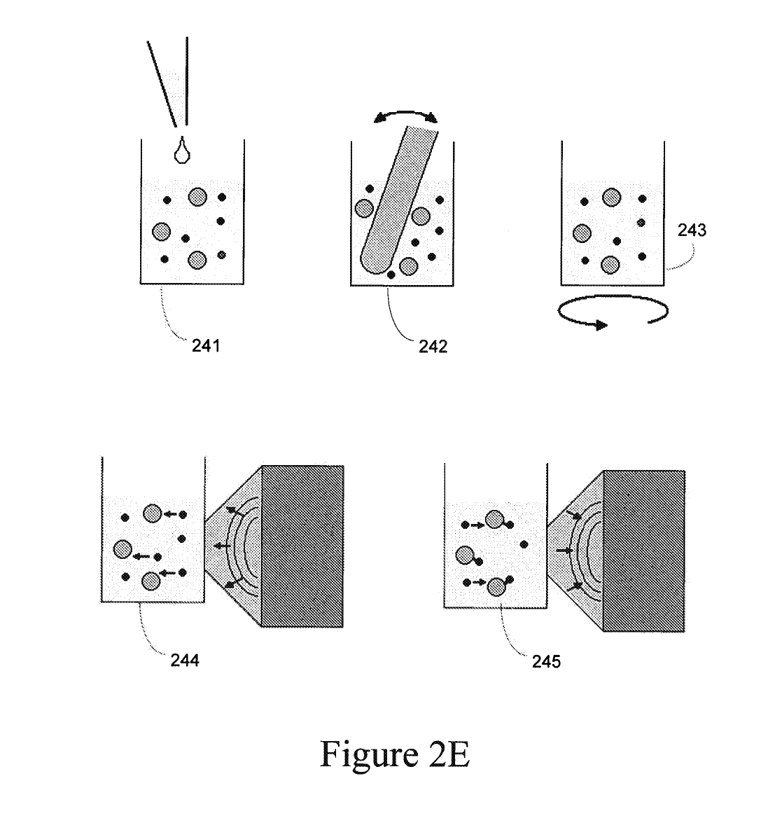

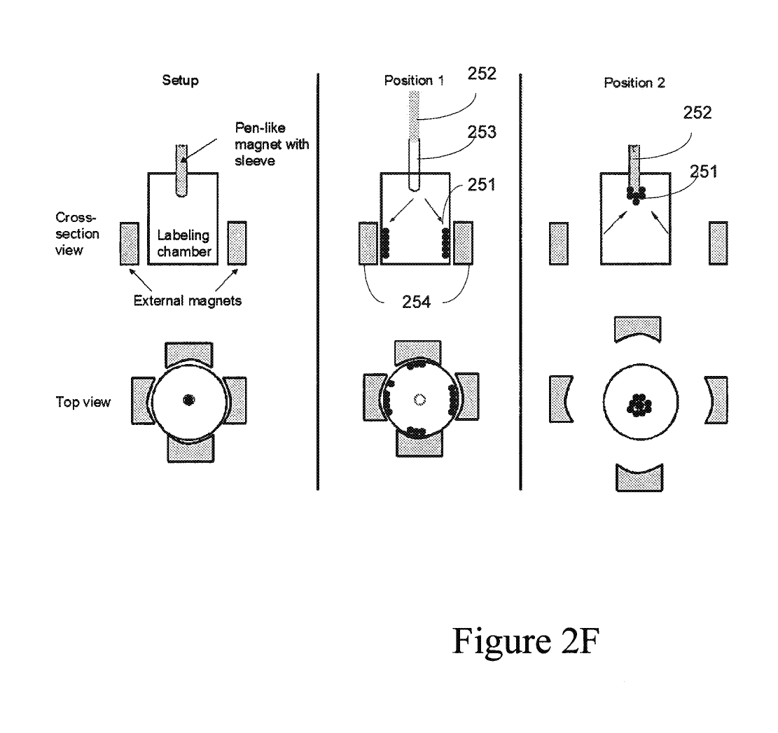

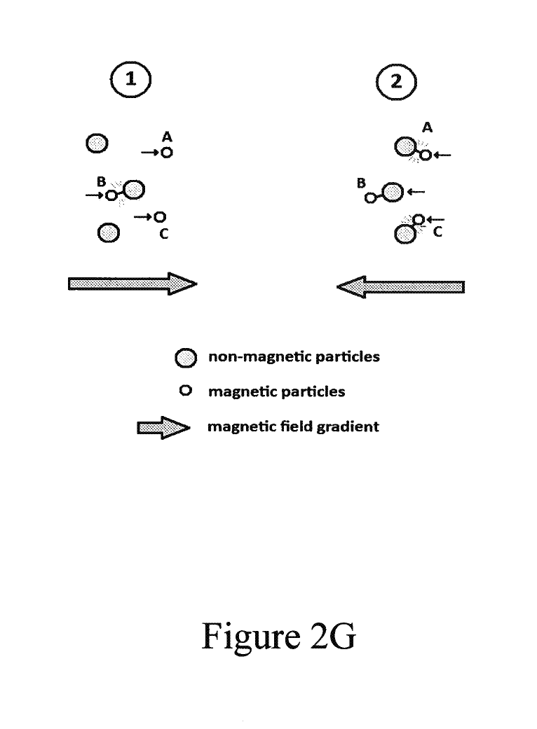

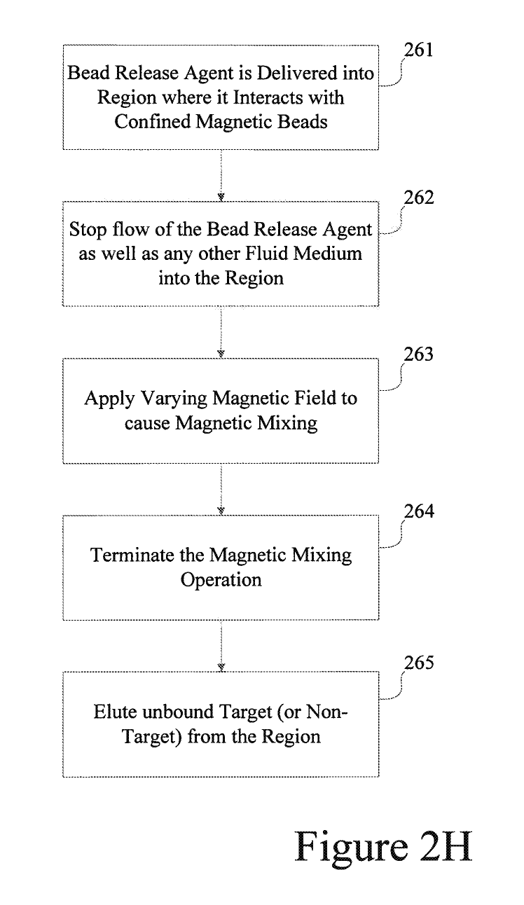

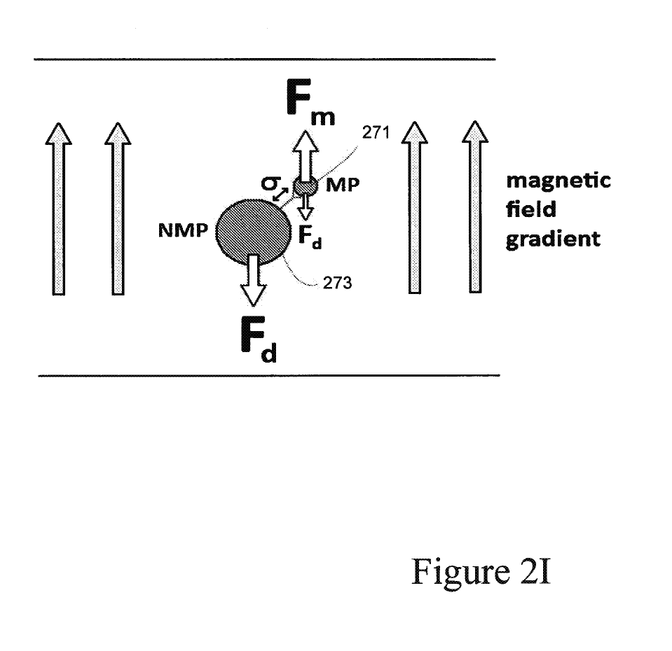

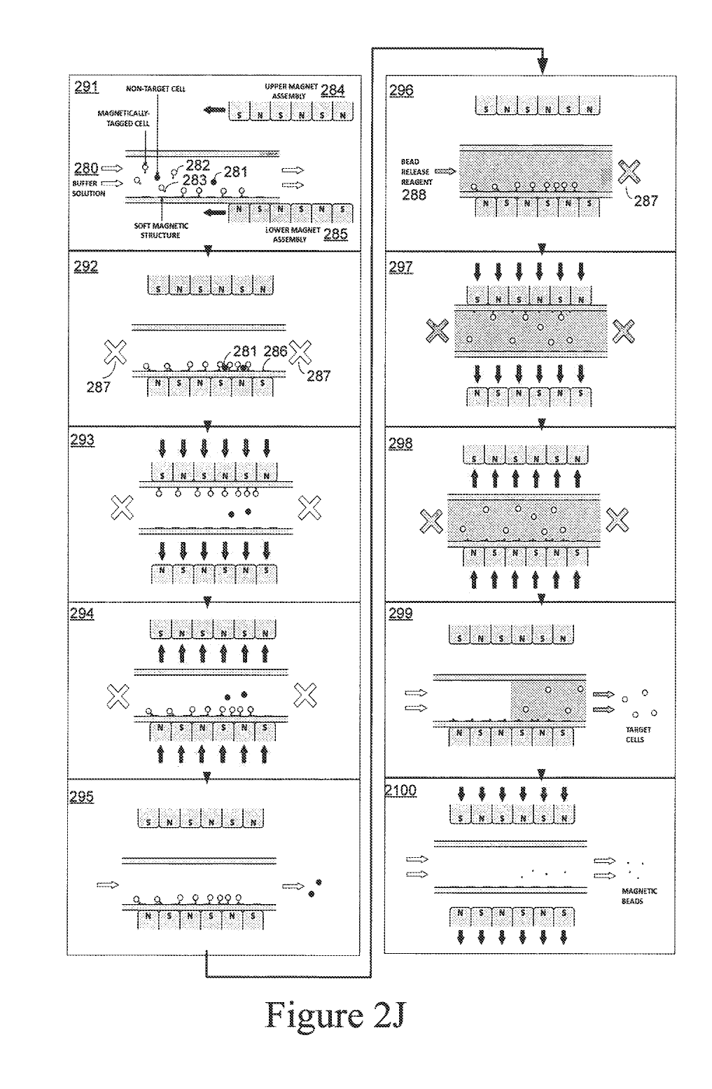

In accordance with embodiments of this invention, magnetic particles are subjected to hydrodynamic forces in order to mix them with a reagent and/or in some cases expose them to shear forces to remove attached species. In certain embodiments, the magnetic particles are exposed such forces while the particles are constrained to a region of a fluidics device such as a chamber or channel; e.g., a sorting region or a pair of reservoirs used for labeling. Typically, though not necessarily, the magnetic particles are suspended within a fluid during the processing; i.e., they are not confined to a particular wall of the device. Examples of systems and methods that provide fluidic mixing of magnetic particles and allow for labeling and/or release of sample species are depicted in FIGS. 2A through 2J, each of which will be described in more detail below.

Typically a single fluidics device (sometimes referred to herein as a “chip”) contains both a sorting station and a one or more mixing stations as described herein. The sorting station separates magnetic from non-magnetic species from a sample. As explained, the functionalized magnetic particles specifically bind with some species (but not all species) of a sample. Thus, the two classes of species may be separated (sorted) based on their affinity for the functionalized magnetic particles. As explained below, two examples of on chip sorting mechanisms are trapping and deflection. The on chip mixing station may be employed to mix magnetic particles with sample, reagent, or other component. The fluidics device is typically, though not necessarily, a unitary device which may be easily moved about as a single portable unit. In some embodiments it is formed from a single solid substrate (e.g., glass or polymer), which may be monolithic and contain multiple stations, channels, ports, and/or other fluidics components. The device may be designed for a single use and therefore be disposable.

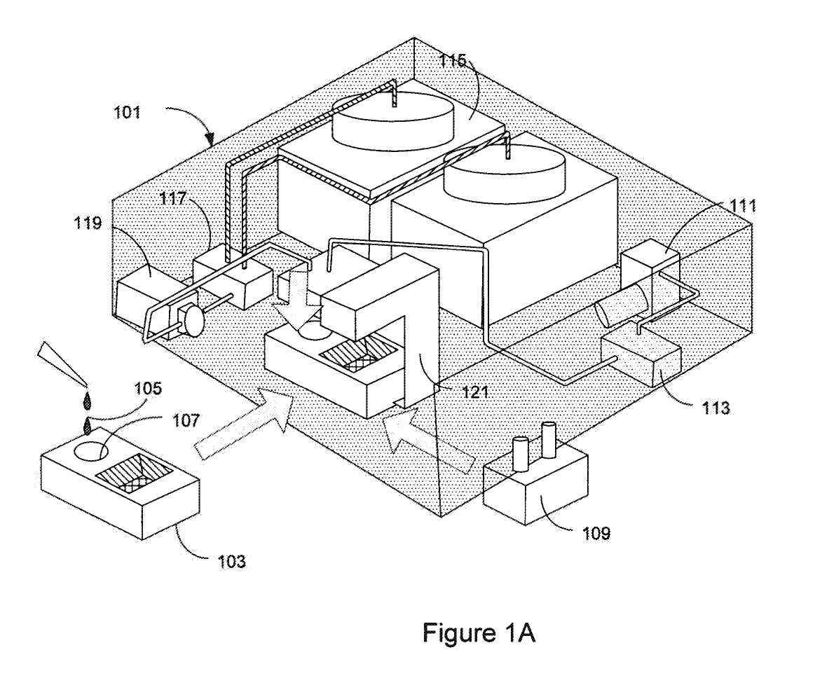

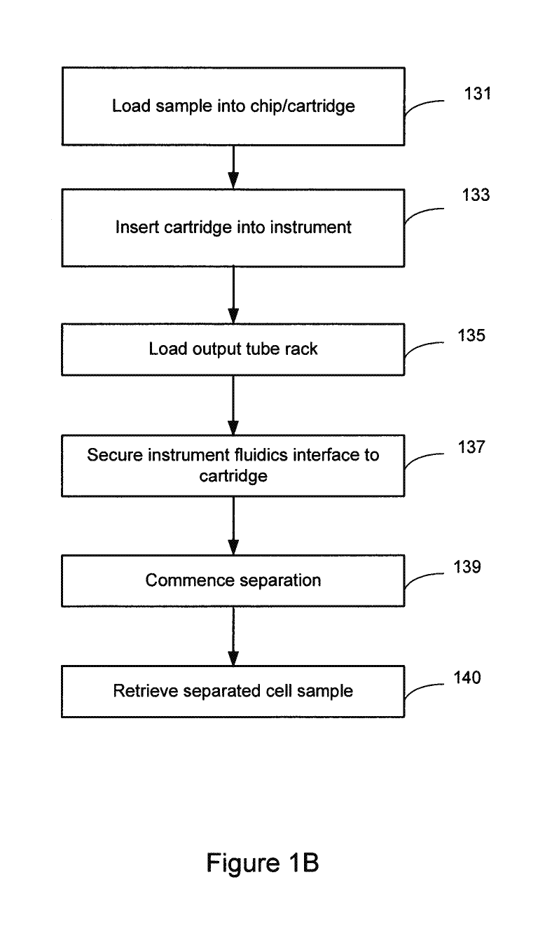

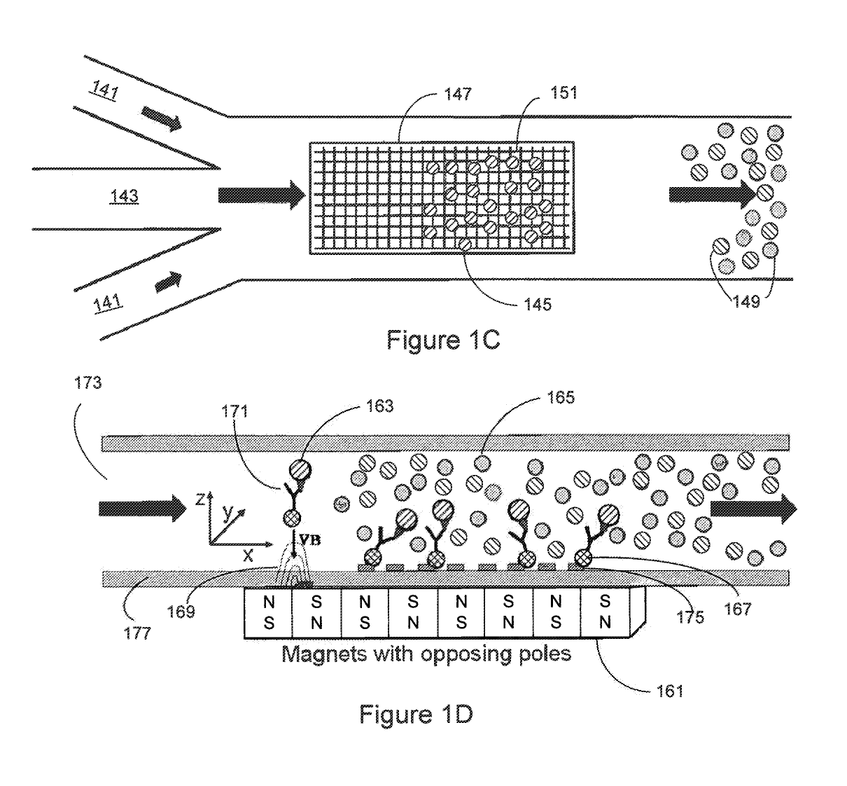

For context an example of a trapping-type magnetic separation system will now be described. FIGS. 1A and 1B illustrate magnetic sorting modules and systems in accordance with certain embodiments. FIG. 1A shows a system 101 that employs disposable fluidics chips or cartridges 103. Each chip or cartridge houses fluidics elements that include a magnetic trapping module. In one mode of operation (positive selection), a sample 105 such as a small quantity of blood is provided to a receiving port 107 of the cartridge and then the cartridge with sample in tow is inserted into a processing and analysis instrument 121. Within the chip, the magnetic particles and the target species (if any) from the sample are sorted and concentrated at the magnetic trapping module. In one embodiment, after sample has been processed in this manner, trapped species may be released and collected in output tubes 109. This may be accomplished by various means including reducing or eliminating the external magnetic field applied to the trapping module or applying a reagent that releases captured species from magnetic particles. Alternatively, or in conjunction, the hydrodynamic force exerted on the magnetic particles may be increased. In the depicted embodiment, a chassis houses the system components including a pressure system (including a syringe pump 111 and a pressure controller 113) that provides the principal driving force for flowing sample through the trapping module. Of course, other designs may be employed using alternative driving forces such as a continuous pump or a pneumatic system. Buffer from buffer reservoirs 115 is also provided to the cartridge under the controlled by a buffer pump 119 and a flow control module 117. In other embodiments, the sorting module employs a continuous flow mechanism in which the magnetic particles are deflected as they flow through the module and are captured in an outlet port oriented to receive only deflected species, i.e., those attached to magnetic particles.

In the depicted embodiment, a pressure system (including a syringe pump and a pressure controller) provides the principal driving force for flowing sample through the trapping module. Of course, other designs may be employed using alternative driving forces such as a continuous pump. Buffer from buffer reservoirs is also provided to the cartridge under the controlled by a buffer pump and a flow control module. Further, as described in more detail herein, various forces may be employed to facilitate mixing of magnetic particles with other components such as samples, release reagents, labeling regents, and other reagents used to process the magnetic particles. Examples of such forces include forces applied by varying external magnetic fields, delivering pneumatic pressure, etc.

In FIG. 1B, an example processing sequence is shown. Specifically, the process begins by loading a sample onto the chip or cartridge before inserting into the instrument, shown as operation 131. Then, in operation 133, the chip is inserted into the instrument to align the external magnet(s), fluidics couplings, and associated apparatus. Thereafter, a collection tube or tubes is also loaded into the instrument (operation 135). Note that in some embodiments the order of loading to the instrument can be varied. Next, a fluidics interface is secured to the chip to ensure leak proof delivery of sample and buffer to the chip in operation 137. Finally, the instrument commences the separation process (139) and separated cell sample may be retrieved by unloading the collection tube (140).

FIGS. 1C and 1D show top and side views of a trapping module in accordance with one embodiment. In a specific example, the depicted trapping module is implemented in a disposable cartridge as shown in FIG. 1A. In the top right diagram of FIG. 1B, a top view of the magnetic trapping module is shown to include a central sample inlet, and two buffer inlets straddling the sample inlet. Buffer delivered from the buffer inlets may prevent contents of the sample from becoming entrained along the edge of the trapping module, and help to stabilize the pressure as well as the flow streams. As shown, a trapping region, which in this embodiment includes a ferromagnetic pattern is formed on a bottom wall of a flow channel. The channel wall on which the pattern is formed may be transparent, semi-transparent or opaque.

As shown, target species 145 are captured on the trapping region. The remaining uncaptured material 149 (or other species) and debris provided with the sample are washed clear of the trapping region because they are not affixed to magnetic particles.

It should be noted that positive or negative trapping schemes may be employed. In a positive trapping scheme as shown in FIGS. 1C and 1D, target species, e.g., 145 and 163, are linked via a linker 171 to the surfaces of the magnetic particles 167 and are thereafter trapped together with the magnetic particle in the trapping region. This effectively purifies and concentrates the target species. In negative trapping embodiments, non target species (rather than target species) are labeled with magnetic particles. Thus, the unlabeled target species continuously flow through the trapping module, while the labeled non-target species are trapped in the trapping region and removed from suspension. This approach purifies the target species, but does so without concentrating them.

A side view of the trapping region in action is depicted in FIG. 1D. As shown, ferromagnetic structures 175 are formed on the inside surface of a lower wall 177 of a flow channel 173. These serve as a magnetic field gradient generating (MFG) structures (described in more detail below). An external magnetic field 169 is typically used as the driving force for trapping the magnetic particles flowing through the fluid medium. The MFG structures 175 may shape the external magnetic field in order to create locally high magnetic field gradients to assist capturing flowing magnetic particles 167. In the depicted embodiment of FIG. 1D, the external magnetic field is provided by an array of permanent magnets 161 of alternating polarity. More generally, the external magnetic field may be produced by one or more permanent magnets and/or electromagnets. In some embodiments, a collection of magnets such as those shown in FIG. 1D are movable, individually or as a unit, in order to dynamically vary the magnetic field applied to the trapping region.

In certain embodiments, the magnetic field is controlled using an electromagnet. In other embodiments, permanent magnets may be used, which are mechanically movable into and out of proximity with the sorting station such that the magnetic field gradient in the sorting region can be locally increased and decreased to facilitate sequential capture and release of the magnetic particles. In some cases using an electromagnet, the magnetic field is controlled so that a strong field gradient is produced early in the process (during capture of the magnetic particles) and then reduced or removed later in the process (during release of the particles).

As shown in the example of FIG. 1D, the magnetic particles are coated with one or more molecular recognition elements 171 (e.g., antibodies) specific for a marker of a target cell 163 or other target species to be captured. Thus, one or more magnetic particles 167, along with a bound cell or other target species 163, flow as a combined unit into the trapping module. For large target species having many exposed binding moieties (e.g., mammalian cells), it will be common to have multiple magnetic particles affixed.

In some embodiments, the trapping region is relatively thin but may be quite wide to provide relatively high throughput. In other words, the cross-sectional area of the channel itself is relatively large while the height or depth of the channel is quite thin. The thinness of the channel may be defined by the effective reach of the magnetic field which is used to attract the magnetic particles flowing through the trapping region in the fluid medium.

Various details of fluidics systems suitable for use with this invention are discussed in other contexts in the description of flow modules in U.S. patent application Ser. No. 11/583,989 filed Oct. 18, 2006 and U.S. Provisional Patent Application No. 61/037,994 filed Mar. 19, 2008, both of which are incorporated herein by reference in their entireties and for all purposes. Examples of such details include buffer composition, magnetic particle features, external magnet features, ferromagnetic materials for MFGs, flow conditions, sample types, integration with other modules, control systems for fluidics and magnetic elements, binding mechanisms between target species and magnetic particles, etc. Generally, in a magnetic trapping module the applied external magnetic field will be relatively higher (considering the overall design of the module) than that employed in a continuous flow magnetic flow sorter of the type described in U.S. patent application Ser. No. 11/583,989. In either case, the magnetic force exerted on target species should be sufficiently greater than the hydrodynamic drag force in order to ensure that the target species (coupled to magnetic particles) are deflected or captured and held in place against the flowing fluid.

In a typical example, the magnetic trapping process proceeds as follows. First, a sample such as a biological specimen potentially containing the target material, which may be cells, parts of cells, protein, or smaller material, are labeled with small magnetic particles coated with a capture moiety (e.g., an antibody) specific for the surface marker of the target material. This labeling process may take place on or off the microfluidic sorting device. In accordance with certain embodiments, the labeling is performed on the same device in a pre-processing module. After this labeling, the sample is flowed into the sorting station (comprising a trapping or deflection region) with or without concurrently flowing buffer solution. Buffer may be delivered through one or more inlets and sample through one or more others. The sorting station is energized with an external magnetic field to deflect or hold the magnetically labeled target cells or other species against the hydrodynamic drag force exerted by the flowing fluid. This occurs while continuously eluting the un-labeled non-target species. As explained above, the magnetic field is typically applied by an external magnet positioned proximate the sorting station. In the trapping embodiments, after most or all of the sample solution has flowed clear of the sorting station, the magnetic components are released. This may be accomplished by any of a number of different mechanisms including some that involve modifying the magnetic field gradient and/or increasing the hydrodynamic force. For example, the magnetic field in the chamber may be reduced, removed, or reoriented and concurrently the sample inlet flow is replaced with release agent (for releasing the captured species) and/or buffer flow. Ultimately the previously immobilized magnetic components, or just their captured species (now purified), flow out of the chamber in a buffer solution. The purified sample component comprising the target species may then be collected at an outlet of the sorting chamber, which, in some configurations may be located directly downstream from the trapping chamber.

It should be understood that embodiments of the invention are not limited to analysis of biological or even organic materials, but extend to non-biological and inorganic materials. Thus, the apparatus and methods described herein can be used to screen, analyze or otherwise process a wide range of biological and non-biological substances in liquids. The target and/or non-target species may comprise small or large chemical entities of natural or synthetic origin such as chemical compounds, supermolecular assemblies, proteins, organelles, fragments, glasses, ceramics, etc. In certain embodiments, they are monomers, oligomers, and/or polymers having any degree of branching. They may be expressed on a cell or virus or they may be independent entities. They may also be complete cells or viruses themselves.

The magnetic capture particles employed in separations of this invention may take many different forms. In certain embodiments, they are superparamagnetic particles or nanoparticles, although in some cases they may be ferromagnetic or paramagnetic. As a general proposition, the magnetic particles should be chosen to have a size, mass, and susceptibility that allow them to be easily diverted from the direction of fluid flow when exposed to a magnetic field in microfluidic device (balancing hydrodynamic and magnetic effects). In certain embodiments, the particles do not retain magnetism when the field is removed. In a typical example, the magnetic particles comprise iron oxide (Fe₂O₃ and/or Fe₃O₄) with diameters ranging from about 10 nanometers to about 100 micrometers. However, embodiments are contemplated in which even larger magnetic particles are used.

The magnetic particles may be coated with a material rendering them compatible with the fluidics environment and allowing coupling to particular target components. Examples of coatings include polymer shells, glasses, ceramics, gels, etc. In certain embodiments, the coatings are themselves coated with a material that facilitates coupling or physical association with targets. For example, a polymer coating on a micromagnetic particle may be coated with an antibody, nucleic acid sequence, avidin, or biotin.

One class of magnetic particles is the nanoparticles such as those available from Miltenyi Biotec Corporation of Bergisch Gladbach, Germany. These are relatively small particles made from coated single-domain iron oxide particles, typically in the range of about 10 to 100 nanometers diameter. They are coupled to specific antibodies, nucleic acids, proteins, etc. Magnetic particles of another type are made from magnetic nanoparticles embedded in a polymer matrix such as polystyrene. These are typically smooth and generally spherical having diameters of about 1 to 5 micrometers. Suitable beads are available from Invitrogen Corporation, Carlsbad, Calif. These beads are also coupled to specific antibodies, nucleic acids, proteins, etc.

As indicated, aspects of this invention pertain to on chip mixing of magnetic particles which may be suspended in a fluidics medium. The mixing involves exposing the magnetic particles to hydrodynamic forces which may originate from various sources. Examples of such sources include pneumatic or hydraulic sources, varying external magnetic and/or electric fields, mechanical stirring, and gravitational fields (e.g., caused artificially by rotational forces). Regardless of the origin of the hydrodynamic force, the magnetic particles are typically confined to a particular region of a fluidics device during the processing. Thus, the particles are typically not subjected to a sorting process in which a magnetically bound portion of a sample is separated from the remainder of the sample. The two following sections present specific examples of pre-separation labeling of sample species and post-separation release of such species.

Pre-Separation Processing

This aspect of the invention pertains to ways to insure that the target or non-target components of a sample become “labeled” with magnetic beads as appropriate. This labeling operation is performed upstream (prior to) the trapping/separating stage in which the magnetic particles are captured and held stationary in a flowing fluid medium.

As explained, the magnetic particles will have a surface functional group that has a specific affinity for either the target or non-target species. Thus, when the magnetic particles come in contact with the relevant species, they bind with those species to form conjugates. An inventive operation pertains to a mechanism for facilitating the binding or conjunction of the magnetic particles with the appropriate species or component from the sample.

Typically, though not necessarily, this pre-sorting treatment is performed in one or more separate chambers or reservoirs located in fluid communication with the trapping region. Such chambers or reservoirs may be located on the same device (chip) as the trapping region or in a separate device or chip. They may have micro fluidic dimensions or even slightly larger dimensions if appropriate. In one example, each of one or more pre-treatment reservoirs has a volume of approximately five milliliters. Typically, the reservoirs may be between 0.5 ml to 10 ml.

The magnetic beads, as well as the sample, and other reagents to facilitate binding are each provided to the reservoir or reservoirs. Note that the magnetic particles may be provided in a functionalized form, in which case it will be unnecessary to provide the other reagents. There, the magnetic particles are moved with respect to the other components in the reservoir(s) to facilitate labeling. This movement is induced by successive application of pneumatic pressure two separate chambers in accordance with certain embodiments. In some embodiments, this movement is induced by a magnetic mixing mechanism of the type described for magnetic particle release as described in a later section. The same mechanisms for facilitating mixing may be employed; e.g., a moving a magnetic field as by, for example, oscillating the field. Other examples of mixing mechanisms include ultrasonic agitation or stirring.

A specific example of a sequence of operations involving a pneumatic labeling operation is presented in FIG. 2A. Initially, as shown in FIG. 2A (block 201), a sample to a first on chip reservoir. This may be a user implemented operation or an automated operation. For example, the user may pipette the sample into the reservoir. Alternatively, in an automated system, an external source of sample delivers a metered amount of the sample via a syringe drive to each of multiple fluidics devices. Other delivery means may be suitable in some embodiments.

Next as illustrated in block 203, magnetic particles (coated with a capture agent such an antibody specific for a target or non-target species in a sample) are added to a second on chip reservoir, which is fluidically connected to the first on-chip reservoir, although it may be temporarily isolated by a closed valve located between the two reservoirs. The magnetic particles may be delivered to the second reservoir manually or automatically as described above for delivery of the sample to the first reservoir.

After filling the reservoirs, each is capped or otherwise sealed in order to prevent the fluids from escaping during on chip mixing. See block 205. This operation is completed after all of the reservoirs have completed the filling process. The capping mechanism may be achieved using a double edge seal, which is integrated into the design of the caps over each well (see FIG. 2I).

Next as illustrated in a block 207, a valve between the first and second reservoirs is opened to allow the fluids in the two reservoirs to mix. In an alternative embodiment, the two reservoirs are not fluidically isolated during the sample and magnetic particle filling operations. In such embodiments, operation 207 is unnecessary.

At this point, the contents of the reservoirs is mixed by pneumatically pushing the contents back and forth between the two reservoirs. See block 209. This mixing may facilitate labeling of particular sample species with the magnetic particles. Alternatively it may facilitate some other pre-sorting process such as contacting a sample with a buffer. In any case, the pneumatic pressure and/or vacuum is sequentially applied to the two reservoirs so that the contents are driven toward one or the other of the reservoirs at any given time.

Finally, as illustrated in block 211 of FIG. 2A, the mixing operation is completed and the labeled species are delivered to a magnetic sorting station downstream from the mixing region. This may be accomplished by opening a valve between a downstream separation station and the reservoirs. Thereafter a driving force is applied to move the labeled and unlabeled portions of the sample into the separation region.

FIG. 2B is a schematic drawing showing a pneumatic system for controlling operation of magnetic separation chip 212 having a trapping region 213 together with the upstream labeling reservoirs as well as a bead release system 214.

In the depicted embodiment, the pneumatic system connected to wells on the chip includes four components per well: a pump, a proportional valve, a switching valve, and a pressure transducer. This arrangement is duplicated for each well, although the pump may be common for all wells. The two valves (proportional and switching) respectively serve the purpose of metering the air pressure at the wells, and venting the wells to atmosphere. By venting the wells to atmosphere, residual air pressure in the reservoirs is released immediately, stopping fluid flow in the chip instantly. The pressure transducer and proportional valves are, in some embodiments, linked in a closed-feedback loop to maintain a set pressure and flowrate, determined by the current stage of the mixing/separation process.

In FIG. 2B, a single pump 215 shown in the top left corner of the figure provides pressure to four separate reservoirs, a “Buffer” reservoir 216, two sample reservoirs (“S₁” (217) and “S₂” (218)) and a release reagent reservoir (“R”) 219. The pneumatic subsystem associated with each of these reservoirs will now be described.

A buffer subsystem is depicted in dashed lines and includes in addition to the on-chip buffer reservoir a proportionate valve PV1 (220), a switching valve SV1 (221) and a pressure transducer T1 (222). The proportionate valve PV1 (220) opens and closes by degrees dictated by the magnitude of an applied control signal (e.g., electrical voltage) and applies precisely controlled pressure levels to the buffer reservoir 216. The switching valve SV1 (221) has two settings, one allowing direct application of pressure from valve PV1 (220) to the buffer reservoir 216 and another allowing venting from the buffer chamber to atmosphere 223. Pressure transducer T1 (222) measures the pressure applied to the buffer reservoir 216. The measured pressure is provided as feedback to proportionate valve PV1 to maintain a desired pressure in the buffer reservoir. Note that the pressure is directly proportional to the flow rate, which is the parameter of most importance in the fluidics system.

In some embodiments, buffer is metered into sample reservoirs S₁ 217 and S₂ 218 through an on chip valve V1 224 in an open position. After a sufficient amount of buffer is delivered to the sample chambers, valve V1 224 is closed and the sample is mixed in a manner as set forth below. This approach may be particularly appropriate in designs where the chip is supplied with pre-packaged reagents.

In certain embodiments, buffer is not added to the sample reservoir prior to mixing. In such cases, buffer may still be metered into sample reservoirs, but only to rinse the sample once the mixing is performed and the sample is flowed into the trapping region.

Buffer may also be delivered to the edges of the trapping region via lines 225 shown along the edge of the separation chip. During a sorting process, this is performed in conjunction with delivering the sample from reservoirs S₁ and S₂ to the trapping region.

Pressure to sample reservoirs S₁ and S₂ is provided from the pump via various fluidic components depicted on the dotted lines. Sample reservoir S₁ (217) has associated proportionate valve PV3, switching valve SV3 and pressure transducer T3. Sample reservoir S₂ (218) has associated proportionate valve PV2, switching valve SV2 and pressure transducer T2. These act on reservoirs S₁ and S₂ in the same manner as elements PV1, SV1, and T1 act on the Buffer reservoir 216.

Mixing of the sample may be accomplished by passing the sample (and associated fluidic components) back and forth between reservoirs S₁ and S₂ through the hatched path 226 joining them. This is performed by alternately applying pressure to each reservoir while a valve V2 remains shut. After a sufficient number of cycles (e.g., about 10-500), the sample is sufficiently mixed and can be supplied to the trapping region to effect sorting.

Note that the components applied sample reservoirs include, in addition to sample, magnetic particles and optionally an affinity binding reagent (e.g., an antibody). After mixing, the beads are coupled to target species via the binding reagent, which is coupled to the bead surface. As indicated buffer is supplied to the sample reservoirs from the buffer reservoir. Prior to mixing, a user may apply the sample, the magnetic particles and the optional binding reagent to the sample reservoirs. The user may also deliver buffer to the Buffer reservoir 216 and a bead release agent to a bead release reservoir (“R”) 219. Each of these components may be provided by, e.g., pipetting. Thereafter, a cap is applied to seal each reservoir and provide a port for delivering pneumatic pressure. An example of a reservoir and cap design is presented in FIGS. 2C and 2D.

After the sample is mixed sufficiently, valve V2 is opened and the sample flows into the trapping region concurrently with buffer. Different collection chambers may be used depending on whether the selection method is positive or negative. In a positive selection, target particles are labeled, trapped, and collected. In a negative selection, target particles are not labeled or trapped and are collected at the outlet after the non-target species are trapped. A negative selection may be useful to isolate unknown substances in a sample by eliminating known substances. If positive selection is employed (i.e., selection for target species on the magnetic particles), valve V4 is closed and valve V3 is opened to allow non-target sample components to be collected in a chamber C−. If, on the other hand, negative selection is employed (i.e., non-target species bind to the magnetic particles), valve V4 is opened and valve V3 is closed to allow the target components from the sample to be collected in a chamber C+.

In certain embodiments, multiple trapping chambers may be connected in series to effectively concentrate a target species. In these embodiments, a first trapping chamber may trap magnetic particles with labeled species along with other undesired species. The trapped material may be released to a second trapping chamber where the target species is further concentrated by removing more of the undesired species. Through the use of two or more trapping chambers in series, very high purity collection is achieved.

A bead release sub-system 214 of the pneumatic system includes a proportionate valve PV4, a switching valve SV4, and a pressure transducer T4, in addition to the on-chip release reagent reservoir, “R.” In one embodiment, the bead release sub-system accomplishes its function on trapped beads in the trapping region as follows. Initially valve V3 is closed while valve V4 is opened (assuming a positive selection approach is employed). Valve V5 is also opened to allow bead release reagent to flow into the trapping region. After a sufficient amount of reagent flows into the trapping chamber, switching valve SV4 is turned to the vent position and no further reagent flows into the trapping region (temporarily). Then, mixing may be performed within the trapping region. In one embodiment this is accomplished by moving magnets (or arrays of magnets) located above and below the trapping region to alternately attract the magnetic particles to the top and then the bottom of the trapping region. As indicated elsewhere herein, this serves to free some trapped non-specifically bound non-target material from the magnetic particles.

After the magnetic mixing (if performed), buffer or additional release reagent may be flowed through the trapping region in order to flush the unbound target into chamber C+. In other embodiments, one or more additional cycles of reagent contact and optional mixing are performed. In such embodiments, switching valve SV4 is turned to the flow position and additional bead release reagent flows into the trapping region from the release reagent reservoir. After a sufficient amount of reagent is flowed into the region, valve SV4 is again turned to the vent position and further magnetic mixing may be performed. In some embodiments, multiple cycles (e.g., 4 cycles) of reagent contact and mixing are performed. After each cycle, additional target material is captured in chamber C+.

In certain embodiments, all components of shown within the dashed line rectangle labeled “Separation Chip” reside on a single unitary substrate such as an injection molded polymeric material (e.g., a polypropylene). A cap covers all or some portion of the substrate including at least one (and usually all) of the reservoirs.

Note that the depicted chip includes a bubble trap (“BT”) 227 on the release reagent and sample inlet lines to the trapping region. In some embodiments, the bubble trap comprises a single membrane that spans two separate channels to capture bubbles on both the reagent and sample lines as shown.

In a typical embodiment, the fluidics system applies a relatively low pressure to drive sample, buffer, and/or other fluid through the fluidics chip. For many applications and designs, a pressure in the range of 0.05 psi to 10 psi is appropriate. For example, sample mixing may be accomplished using a pressure of about 0.1 to 1 psi applied (alternately) to the two sample reservoirs. For buffer flushing, however, a higher pressure may be appropriate, e.g., about 5 psi.

In many designs, the components of the pneumatic system (pump, valves and transducers) are all commercially-available, off-the-shelf components that can be acquired at relatively low cost from vendors such as Hargraves Technology Corporation (Mooresville, N.C.), and Clippard Instrument Laboratory (Cincinnati, Ohio). In various embodiments, the entire pneumatic system may be replaced with an equivalent system delivering a set amount of flow utilizing a different force, such as hydraulic, magnetic or electrical force.

FIGS. 2C and 2D depict reservoirs for holding samples, buffers, reagents, and the like in a fluidics chip. The depicted reservoirs may be used with flow delivery systems such as that depicted in FIG. 2B. The depicted reservoir has a downward sloping bottom surface that funnels toward an exit port 230. It also has a main holding portion 231 and a cap 232 to seal the top of the reservoir from the external environment.

The downward sloping bottom surface facilitates draining liquid including magnetic particles (and possibly other components) through the outlet port 230. It may be generally conically shaped allow other downward sloping shapes may be used as well (e.g., various pyramidal shapes). The main holding portion 231 may be of any suitable shape such as cylindrical, oval, polygonal, etc.

In a specific embodiment, the sample reservoirs are designed with a capacity of 5 mL each, to allow complete transfer of a 5 mL from well to well. In the depicted example, the wells are cylindrical in shape, with an inverted-cone bottom surface. This shape is similar to that of a standard centrifugation tube.

As depicted in FIGS. 2C and 2D, a cap 232 is designed to fit over and seal one or more reservoirs on the chip. The cap is made from a material complementary to the chip substrate material to allow for a leak-proof seal. In one embodiment, the cap is made from a polymeric or elastomeric material. In a specific embodiment, both the chip substrate are polymeric materials, e.g., polypropylene, which may be fabricated by injection molding. In a specific embodiment, the cap is between about 0.8 and 1.5 millimeters thick in the region overlaying the reservoir. In the depicted embodiment, the cap has a dome shape over the reservoir. This may desirably provide an air gap between the upper surface of the liquid in the reservoir and the cap and thereby minimize the amount of liquid that adheres to the cap.

In some cases, the cap and the upper surface of the reservoir may have mating surfaces to facilitate sealing. For example, in the depicted embodiment, the upper surface of the reservoir includes a circumferential lip 233 extending upward from a principal plane of the chip substrate. A complementary groove 234 is provided in the cap to engage the lip and provide a seal for preventing ingress and egress of liquid.

Coupling of the flow delivery system to the chip is achieved using a simple gasket 235 (e.g., an elastomeric o-ring) that creates an airtight seal with a rigid manifold 236 in the system apparatus. See FIG. 2D. A port in the manifold aligns with a port in the upper surface of the cap (the dome region above the manifold) to permit direct application of pressure 237 from the system to the fluid in the reservoir.

FIG. 2E presents, in simplified conceptual fashion, alternative non-magnetic mixing methods. In the top left illustration 241, a sample is loaded into a mixing chamber with affinity ligand-tagged magnetic beads. In the top center illustration (242), a stirring structure is used to physically mix the beads and sample. In the top right illustration (243), the mixing chamber is vibrated in an oscillating fashion to cause mixing of the contents to occur. In the two bottom illustrations (244 and 245), an ultrasonic transducer sends pressure waves into the mixing chamber to produce relative movement of the beads to the target species. The relative movement causes multiple collisions between beads and target species and thereby induces binding.

FIG. 2F illustrates a center-pole magnet mixing station (see left illustrations) and method that may be implemented on or off a fluidic device in which magnetic-mediated sorting takes place. In the depicted mixing system, the sample is loaded into the labeling chamber with affinity-tagged magnetic beads 251. In Position 1 (center illustrations), a pen-like magnet 252 is withdrawn from the sleeve 253, and external magnets 254 (four shown in this embodiment) are brought in contact or close proximity with the outside surface of the chamber, thereby moving the magnetic beads to the walls of the chamber. In Position 2, the external magnets are withdrawn, and the pen-like magnet 252 is inserted into the sleeve that is immersed in the solution, thus attracting the magnetic beads 251. The relative movement of the beads 251 through the sample-bearing solution induces collisions that enhance binding. Movement between Positions 1 and 2 can be repeated several times as necessary to accomplish complete labeling.

FIG. 2G is a simplified diagram depicting the effects of oscillation of a magnetic field gradient on magnetic particles. This effect may be generated by varying the position of an external magnet or collection of magnets during a mixing process. Initially, magnetic particles A, B, and C are not bound to any target species. As a magnetic field gradient first directs these particle to move from left to right, magnetic particle B comes in contact with a target species and becomes bound thereto. Later, when the direction of the external magnetic force changes so that the magnetic particles tend to move from right to left, particles A and C encounter target species and become bound thereto.

Another pre-separation processing may include washing raw magnetic particles, for example, magnetic particles containing preservatives, with a wash buffer prior to labeling. According to various embodiments, raw magnetic particles may be introduced to a sample well before any sample containing target species is added. The raw magnetic particles may contain preservatives, which is preferably removed before the particles are used to label a target species. A wash buffer may be introduced to a different sample well or the same sample well containing the magnetic particles. Mixing techniques described herein for mixing and labeling samples and for agitating magnetic particles in the sorting chamber may be used to wash the preservatives from the magnetic particles. An external magnet may be used to retain the magnetic particles in the sample well while the wash buffer drains. In other embodiments, the magnetic particles may be allowed to drain into the sorting chamber where they will become trapped by the magnetic field. From the sorting chamber, the magnetic particles may be released into an outlet where it can be collected and re-introduced in a sample well for the labeling process. In still other embodiments, the washed magnetic particles may be returned directly into the sample well.

Post-Separation Processing

The post separation operations described here involve primarily methods for releasing target species from magnetic particles that have been trapped in a trapping station or otherwise separated in a sorting station. In a typical scenario, at the end of a trapping operation, the only sample species that remain in the trapping region are bound to magnetic particles. For many applications, it is important to separate the captured species from the magnetic particles prior to further processing.

In the post separation operations described here, some mechanism for releasing the bound species from the magnetic particle is employed. Various binding and release systems are available. These include, for example, release reagents that (1) digest a linkage chemically coupling the magnetic bead to the captured species, (2) compete with chemical or biochemical linkage mechanisms for binding with the captured species, and (3) cleaving the linkage with a secondary antibody.

The advantages of magnetic mixing include (1) exposing the magnetic bound particle pair to more release agent in the solution and (2) exposing the magnetic bound particle pair to increased fluidic drag to provide stress on the linkage between the magnetic particle and its non-magnetic payload. This increases the probability of dissociation.

In accordance with some embodiments, a bead release reagent will be introduced into the trapping region, and then mixed with the magnetic particles to facilitate releasing the bound species. A flow chart shown in FIG. 2F depicts a typical particle release process, which process may be performed iteratively. Initially, as depicted in FIG. 2F, the bead release agent is flowed into the trapping region where it may interact with the captured magnetic beads in block 261. The next operation in the process flow involves stopping the flow of the bead release agent as well as any other fluid medium into the trapping region, as shown in block 262. This allows the next operation 263, a magnetic mixing, to be performed without elution of bound target species. In certain embodiments, the magnetic mixing involves moving the magnetic beads from the bottom toward the top of the trapping region or vice or versa. This causes the magnetic particles to move back and forth sequentially. Typically this is accomplished by reducing the magnetic field strength at the bottom of the trapping region and concurrently increasing it at the top of the region or vice or versa depending upon whether the magnetic particles where initially trapped on the bottom or the top of the trapping region. Moving of the beads back and forth within the trapping region exposes their payload to the hydrodynamic drag, thereby facilitating release of payload. Further, the motion more effectively exposes the magnetic beads to the bead release agent, without relying on diffusion exclusively.

The magnetic mixing operation may be characterized in terms of various parameters such as the direction of motion, the frequency of the oscillations, the duration of the process, etc. In one example, the beads were moved back and forth at a frequency of 0.15 Hertz for 15 minutes. This frequency and mixing duration are representative of an approximately minimum frequency and maximum mixing period respectively—depending on the size, magnetic field saturation strength of the beads, and other factors such as the fluid viscosity, the frequency can be varied across a wide range. In the case of relatively large magnetic beads (e.g. 4.5 μm diameter), the frequency may be increased to ˜1 Hertz, and the mixing period reduced proportionally to about 2 minutes.

The next operation of the process involves terminating the magnetic mixing operation in block 264 by, e.g., putting the strong magnetic field back into position at the bottom of the trapping region channel to thereby attract and capture the magnetic particles again. Presumably, at this point the particles are now largely unbound, i.e., separated from their target (or non-target) species.

A subsequent operation 265 involves flushing out the unbound target (or non-target) from the trapping region. This may involve flowing fresh bead release agent or other fluid through the trapping region while the magnetic particles remain affixed to the bottom of the trapping region. This causes elution of the separated species.

While the sequence of five operations depicted above may be sufficient to effectively release and elute all or substantially all the trapped target species in many applications, other applications may require multiple repetitions of operations 262 to 265 to effectively remove all the target species from the trapping region. Regardless of how many repetitions are employed, the very last elution step may involve flowing either a buffer or bead release agent into the trapping region. In all prior operations, the delivery of fresh fluid into the trapping region will typically entail delivery of a bead release agent to facilitate further release of bound target species.

FIG. 2I shows force vectors acting on magnetic beads with bound target species under the influence of a varying magnetic field. As shown, a magnetic field gradient having a vertical direction produces an upward vertical force (Fₘ) on a bound magnetic particle (MP). This tends to move the coupled magnetic particle and bound target species upward against the resistance of hydrodynamic drag in the fluid medium. This resistance is represented as a downward force vector (F_{d}) acting on both the magnetic particle 271 and the non-magnetic particle 273 (NMP the target species). The opposing forces also impart a shear force on the linkage between the magnetic particle and the bound target species, which shear force tends to break apart the linkage between the two particles.

FIG. 2J depicts an apparatus for magnetic particle release and an associated sequence of operation in accordance with certain embodiments. The drawing includes a sequence of cross-sectional views that begin at the top left of the figure and proceed down the left side and then down the right side of the same figure. The depicted general operations include sequentially (a) trapping magnetic particles, (b) cleaning the trapped particles of unbound sample, and (c) releasing the bound species from the trapped magnetic particles.

The depicted apparatus includes an on chip fluidic trapping station which receives a buffer solution 280 and a mixture of labeled and unlabeled sample species. The non-target species 281 are depicted as dark circles while the target species 282 are depicted as light circles. The target species are coupled to magnetic particles 283 which are shown as small dots. The fluidics trapping station includes upstream and downstream valves that can isolate the station so that no fluid enters or leaves the station during certain operations.

The depicted apparatus also includes two groups of external magnets (284 and 285), upper and lower arrays of alternating polarity permanent magnets. These arrays can move with respect to the trapping station with at least two degrees of freedom. First, they can move laterally along the direction of flow of the sample and buffer and second, they can move in an orthogonal direction, toward and away from upper and lower surfaces of the trapping station of the fluidics device. The two arrays of magnets may move independently of one another or coupled together dependently.

The specific sequence of operations shown in FIG. 2J includes sliding magnet insertion while sample flows into the trapping station, separation by magnetic trapping, non-specific species removal wash, and bead release. In the upper left panel 291, a sample is injected into the channel of a trapping station which may include a ferromagnetic grid. While the sample flows into the trapping station, the lower external magnet assembly moves under the grid in the opposite direction opposite the fluid flow. In this example, the upper magnet assembly is fixed relative to the lower magnet assembly so that the two of them move in tandem. As shown, some of the magnetically labeled species become bound to the lower surface of the trapping station during this operation.

Next, as shown in panel 292, the magnet assemblies are fully in position over the capture surface of the trapping station. At this point, all magnetically-labeled species are trapped on the lower surface of the station, which may include a soft magnetic structure 286 (e.g., a ferromagnetic trapping grid). Incidentally, a few non-magnetically-tagged species 281 are also trapped due to non-specific physical entrapment. At the conclusion of this process, valves 287 (shown as “X”s) are closed at the upstream and downstream sides of the station.

Next, as shown in the panel 293, the upper/lower magnet assemblies are translated vertically to bring the upper magnet assembly 284 in contact with the top of the channel. Concurrently, the lower magnet assembly 285 is moved sufficiently far away from the device to release the magnetic beads from the lower surface, e.g., the trapping grid. The magnetically-labeled species move toward the top wall of the channel, leaving the non-magnetically-tagged species free in solution. This is down while the valves remain closed so that little or no fluid enters of leaves the trapping station.

Next as shown in panel 294, the magnet assembly position is reversed to bring the lower magnet assembly back to its original position after trapping. This operation and the previous operation can be repeated one or more times (e.g., multiple times) to ensure that all non-magnetically-labeled species are free in solution within the trapping station. In the depicted embodiment, the valves remain closed during this entire operation. Thereafter, the valves are opened and a buffer solution is pumped through the channel to elute the non-magnetically-tagged species. See panel 295 on the left side of FIG. 2J.

The bead release operations are depicted on the right set of panels in FIG. 2J. As shown, in panel 296, the trapping station is filled with bead release reagent 288 and then the flow is stopped; i.e., the downstream valve 287 is closed when the station contains a sufficient amount of release agent. Next as depicted in the second panel on the right side, the magnet assembly is translated vertically to move the beads from the bottom to the top wall of the channel. As depicted, this is performed in the same manner as discussed previously in the context of freeing non-specifically bound sample species. The upstream and downstream valves 287 are closed. The combined effect of the bead release reagent and the magnetic bead movement relative to the target species releases some of the targets from their magnetic beads into the solution.

Next, as shown in panel 298 on the right side, the magnet assembly position is reversed to bring the lower magnet assembly 285 back to its original position. The valves 287 remain closed. This operation and the previous one can be repeated one or more times to ensure that all the beads are released from their attached targets. Now, with the lower magnet assembly back at the lower position and the beads trapped on the trapping grid, the valves 287 are opened and buffer solution is flowed through the channel, eluting the now free target species. See panel 299 on the right side. Finally, in the depicted embodiment shown in panel 2100, the magnet assembly is moved halfway up to remove the interaction of both external magnetic assemblies, allowing the beads to be eluted from the channel with buffer solution.

In an alternative embodiment, the permanent magnet assemblies are replaced with electromagnets as the external magnets. The magnetic mixing may be implemented by alternating energizing electro magnets on the top and bottom of the trapping region.

In other magnetic mixing embodiments, the magnetic field moves in a direction other than bottom to top and vice or versa. As an example, mixing could be facilitated by moving the beads left and right or front to back within the trapping region so long as there is little or no flow of fluid within the trapping region during this mixing.

Dynamically Varying External Magnetic Fields

In accordance with embodiments of this invention, a dynamically varying magnetic field may be applied to the trapping region during flow of the magnetic particles. This may involve, for example, progressive insertion of a magnetic field over the trapping region during the trapping operation.

This approach has the advantage of reducing or preventing build up of magnetic particles at the leading edge or elsewhere in the trapping region. Generally, a build up has been observed to occur where the magnetic field is strongest, typically at the edge of a permanent magnet used to apply the external magnetic field. As should be clear, such build up can result in under utilization of the trapping region (portions of the trapping region where the magnetic field strength is not great might not capture many or any of the magnetic particles). Further, the clump or pile up of magnetic particles may actually block passage of further magnetic particles to the down stream portions of the trapping region. It may also capture unbound species from the sample and thereby reduce purity of the captured components of the sample.

By using a dynamically varying magnetic field in accordance with this invention, one can produce a relatively evenly dispersed layer of the magnetic particles captured over the trapping region. In some cases, this layer is effectively a monolayer of magnetic particles on the trapping region, although bilayers and the like may be produced depending upon the area of the trapping region and the quantity of sample to be processed. In some cases, the design and application may result in sub-monolayer coverage; i.e., less than the full capacity of the capture surface is utilized.

A relatively uniform distribution of magnetic particles in the trapping region may be useful during post-separation operations such as bead release. The release agent will fill the entire the channel and the uniform spreading of magnetic bound target particles will allow greater access to the magnetic bead bound target particles by the release agent.

The external magnet (or a system of magnets) that is variably positioned during capture of the magnetic particles may be driven by any of a number of different means, some of which will be described below. Further, the external magnet may be a permanent magnet or electromagnet, or multiples of either of these or combinations of permanent and electromagnets.

In accordance with some embodiments of this invention, the position of greatest magnetic field strength is gradually moved over the trapping region during the period of time when particles are flowing into the channel. The direction of movement of the magnetic field during trapping may be, in one embodiment, from a down stream position to an up stream position within the trapping region. In other words, the direction of movement of the magnetic field is opposite that of the direction of the fluid flow in the trapping region. Such embodiments may involve, for example, moving a permanent magnet in a direction from a downstream position to an upstream position underneath the base of a flow channel, particularly the region of the channel comprising the trapping region. Thus, as magnetic particles first enter the trapping region, the leading edge of the permanent magnet is positioned just beyond the downstream edge of the trapping region. Thereafter, as the magnetic particles begin to flow into the trapping region, the leading edge of the permanent magnet is gradually moved upstream and ultimately comes to rest at or near the upstream boundary of the trapping region. In certain embodiments, it reaches its position at about the time when the magnetic particles cease flowing into the trapping region.

In an alternative embodiment, the external magnet moves from the upstream to the downstream positions of the trapping region during capture of the magnetic particles. In other words, the external magnet moves in the same direction as the fluid flow. In this embodiment, as in the previously described embodiment, the duration of the movement of the external magnet should correspond, at least roughly, to the period of time during which magnetic particles are flowing through the trapping region. One specific embodiment employs a downstream movement of a magnet to sequentially capture and release and capture and release . . . the same particles in order to remove non-specifically bound sample species from the magnetic particles.

As indicated, control of the repositioning of the magnetic field within the trapping region can be accomplished by various mechanisms. In a first embodiment, this is accomplished by moving a magnetic field producer (e.g., a permanent magnet) over one surface of the trapping region (typically outside the channel) during the passage of magnetic particles through the trapping region. In another embodiment, the external magnet is an electromagnet which moves along the trapping region (same as the permanent magnet) during the flow of magnetic particles into the trapping region. Optionally, the position of the magnetic field produced by the electromagnet can be controlled by other means such as mechanically moving some or all of the electromagnet's coils during the trapping period.

In another embodiment, the dynamic repositioning of the magnetic field during trapping is accomplished by sequential insertion of a series of external magnets, each of relatively small size with respect of the size of the trapping region. In one embodiment, the magnets are permanent magnets. In a specific embodiment, these permanent magnets are arranged in alternating polarities (e.g., a first magnet has its south pole oriented toward the trapping region, a second magnet has its north pole oriented toward the trapping region, a third magnet has its south pole oriented toward the trapping region, a fourth magnet has its north pole oriented toward the trapping region, etc.). FIG. 1B shows an example of such arrangement of permanent magnets.

Typically, in embodiments involving sequential insertion of the plurality of magnets, the magnets are arranged along the axial flow direction. In one example, the number of magnets is about 5 to 50. In a specific embodiment, about 20 separate permanent magnets are employed and arranged in alternating polarities, each having a width (dimension along the axial flow direction) of approximately 0.5 to 10 millimeters (e.g., 1.5 millimeters). More generally, the width of the individual permanent magnets is determined, at least in part, by the axial length of the trapping region and the number of magnets to be inserted.

In a typical embodiment, the first inserted magnet is the most downstream magnet and then progressively the upstream magnets are inserted during the course of the introduction of magnetic particles into the trapping chamber. In an alternative embodiment, the sequence of insertion can be reversed such that the first inserted magnet is the leading upstream position magnet, the second inserted magnet is the next successive downstream positioned magnet, etc.

Those of skill in the relevant art will understand that there are numerous other actuating mechanisms that could be used to mechanically, electrically, and/or electromechanically position magnets within the domain of a trapping region during fluid flow. Examples include solenoid drivers, electrical motors, pneumatic drives, hydraulic drivers, and the like.

The timing of the insertion of the external magnet(s) into the trapping region, in typically embodiments, corresponds at least roughly to the time period during which magnetic particles flow through the trapping region. In other words, the movement of the external magnet with respect to the trapping region may begin at about the same time that magnetic particles are introduced to the trapping region and end at about the same time when the last magnetic particles leave the trapping region. It may be useful to characterize this duration (the total time in which the magnetic particle bearing solution flows through the trapping region) as a “separation period.” Thus, in some embodiments, this period corresponds, at least roughly, to the period of time during which the external magnetic field is dynamically varied in the trapping region (e.g., the time during which external magnets are moved with respect to the trapping region). In other cases, however, the magnetic field will be fully developed in the trapping region for some time prior to the end of the separation period. In either case, the movement of the external magnetic field with respect to the trapping region may be smooth and continuous or stepped and discontinuous, as appropriate for the particular application.

Typically, the magnetic field when fully applied to the trapping region at the end of the separation period may be maintained for a further period of time to retain the magnetic particles in the trapping region for subsequent processing such as washing, release of captured target agent, etc.

Processing Trapped Species

In some embodiments, trapped species will be released from their associated magnetic particles in while confined to a trapping region. As mentioned, various mechanisms may be employed for this purpose. One approach involves applying a bead release agent to the trapped magnetic particles. Such agents may act by cleaving a chemical linker between the beads and the captured species or by competitively binding a linking species. Of course, other cleaving or release agents may be employed as will be understood by those of skill in the art.

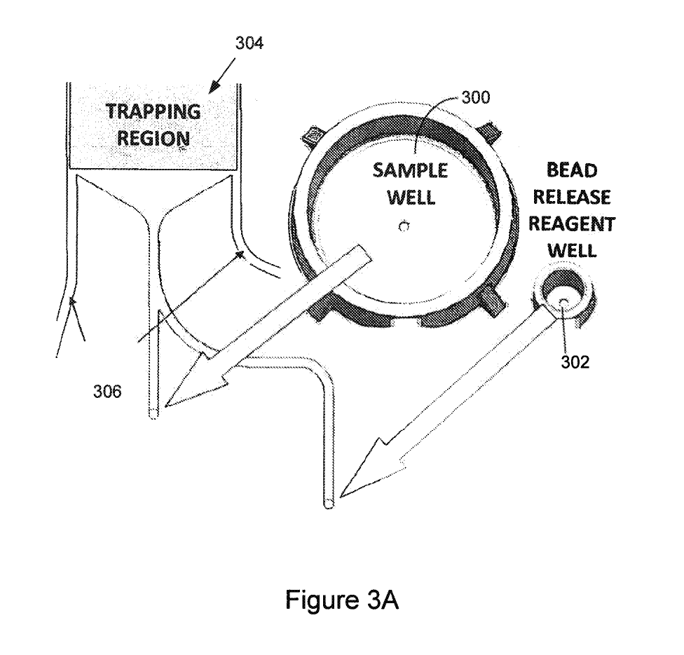

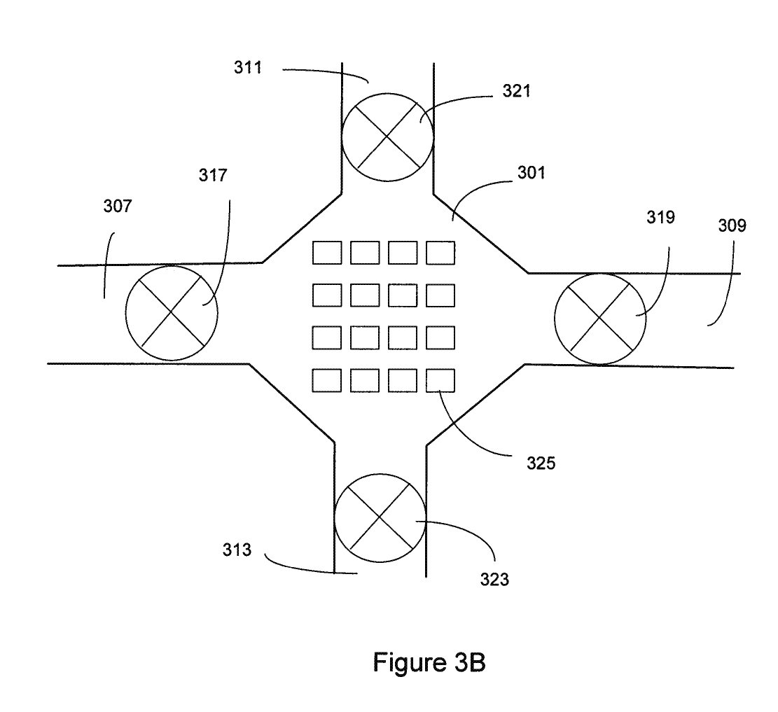

FIG. 3A depicts a fluidics input for a sample well 300 and a bead release reagent well 302. During a separation process, sample is pumped from sample well 300 into a trapping region 304. Once separation process is complete, bead release reagent is pumped from the release reagent well 302 into the trapping region 304. To elute the released targets, buffer can be pumped in from either of the input wells, or from wall buffer inlets 306. The pumping action in all cases can be achieved using, e.g., either a gas (such as air) or liquid (such as buffered water).

Trapped target species may be simply concentrated, purified and/or released as described. Alternatively they can be further analyzed and/or treated. FIG. 3B shows the structure of a magnetic trap 301 disposed in a fluidics device 305 for post-capture treatment of captured species. As shown, trap 301 includes an inlet line 307 for receiving a raw sample stream and an outlet line 309. Trap 301 also includes auxiliary lines 311 and 313 for providing one or more other reagents. Each of lines 307, 309, 311, and 313 includes its own valve 317, 319, 321, and 323, respectively. Within trap 301 are various trapping elements 325. These may be ferromagnetic elements that shape or deliver a magnetic field, etc.

While a magnetic field or other capturing stimulus is applied to the trap features 325, the particles flowing into trap 301 are captured. After a sufficient number of particles are captured (which might be indicated by simply running a sample stream through device 305 for a defined period of time), valves 317 and 319 are closed. Thereafter, in one embodiment, valves 321 and 323 are opened, and a buffer is passed from line 311, through trap 301, and out line 313. This serves to wash the captured particles. After washing for a sufficient length of time, the washed particles may be recovered by eluting (by e.g., removing an external magnetic or electrical field while the buffer continues to flow), by pipetting from trap 301, by removing a lid or cover on the trap or the entire device, etc. Regarding the last option, note that in some embodiments the devices are disposable and can be designed so that the top portion or a cover is easily removed by, e.g., peeling. In any of these cases, the species may be released from their magnetic particle labels prior to further processing by one or more the techniques described above.

In another embodiment, the particles that have been captured and washed and optionally released in the trap as described above are exposed to one or more markers (e.g., labeled antibodies) for target species in the sample. Certain tumor cells to be detected, for example, express two or more specific surface antigens. To detect these tumors, more than one marker may be used. This combination of antigens occurs only in very unique tumors. To detect the presence of such cells bound to magnetic particles, valves 317 and 323 may be closed and valve 321 opened after capture in trap 301 is complete. Then a first label is flowed into trap 301 via line 311 and out via line 309. Some of the label may bind to immobilized cells in trap 301. Thereafter, valve 321 is closed and valve 323 is opened and a second label enters trap 301 via line 313. After label flows through the trap for a sufficient length of time, the captured particles/cells may be washed as described above. Thereafter, the particles/cells can be removed from trap 301 for further analysis or they may be analyzed in situ. For example, the contents of trap 301 may be scanned with probe beams at excitation for the first and second labels if such labels or fluorophores for example. Emitted light is then detected at frequencies characteristic of the first and second labels. In certain embodiments, individual cells or particles are imaged to characterize the contents of trap 301 and thereby determine the presence (or quantity) of the target tumor cells. Of course various target components other than tumor cells may be detected. Examples include pathogens such as certain bacteria or viruses.

In another embodiment, nucleic acid from a sample enters trap 301 via line 307 and is captured by an appropriate mechanism (examples set forth below). Subsequently, valve 317 is closed and PCR reagents (nucleotides, polymerase, and primers in appropriate buffers) enter trap 101 via lines 311 and 313. Thereafter all valves (317, 319, 321, and 323) are closed and an appropriate PCR thermal cycling program is performed on trap 301. The thermal cycling continues until an appropriate level of amplification is achieved. Subsequently in situ detection of amplified target nucleic acid can be performed for, e.g., genotyping. Alternatively, the detection can be accomplished downstream of trap 301 in, e.g., a separate chamber which might contain a nucleic acid microarray or an electrophoresis medium. In another embodiment, real time PCR can be conducted in trap 301 by introducing, e.g., an appropriately labeled intercalation probe or donor-quencher probe for the target sequence. The probe could be introduced with the other PCR reagents (primers, polymerase, and nucleotides for example) via line 311 or 313. In situ real time PCR is appropriate for analyses in which expression levels are being analyzed. In either real time PCR or end point PCR, detection of amplified sequences can, in some embodiments, be performed in trap 301 by using appropriate detection apparatus such as a fluorescent microscope focused on regions of the trap.

For amplification reactions, the capture elements 325 capture and confine the nucleic acid sample to reaction chamber 301. Thereafter, the flow through line 307 is shut off and a lysing agent (e.g., a salt or detergent) is delivered to chamber 301 via, e.g., line 311 or 313. The lysing agent may be delivered in a plug of solution and allowed to diffuse throughout chamber 101, where it lyses the immobilized cells in due course. This allows the cellular genetic material to be extracted for subsequent amplification. In certain embodiments, the lysing agent may be delivered together with PCR reagents so that after a sufficient period of time has elapsed to allow the lying agent to lyse the cells and remove the nucleic acid, a thermal cycling program can be initiated and the target nucleic acid detected.

In other embodiments, sample nucleic acid is provided in a raw sample and coupled to magnetic particles containing appropriate hybridization sequences. The magnetic particles are then sorted and immobilized in trap 301. After PCR reagents are delivered to chamber 301 and all valves are closed, PCR can proceed via thermal cycling. During the initial temperature excursion, the captured sample nucleic acid is released from the magnetic particles.

The nucleic acid amplification technique described here is a polymerase chain reaction (PCR). However, in certain embodiments, non-PCR amplification techniques may be employed such as various isothermal nucleic acid amplification techniques; e.g., real-time strand displacement amplification (SDA), rolling-circle amplification (RCA) and multiple-displacement amplification (MDA). Each of these can be performed in a trap such as chamber 301 shown in FIG. 3B.

Example Magnetic Trapping Structures

Most fundamentally, a trapping station is defined by the boundaries of a region or channel in a fluidics device. Fluid flows through the trapping station and encounters a magnetic field generated by one or more external magnets proximate the trapping station. In addition, a trapping station may optionally employ a magnetic field gradient generator (MFGs). MFG elements (e.g., strips, pins, dots, grids, random arrangements, etc.) shape the external magnet field to produce a locally high magnetic field gradient in the trapping station.

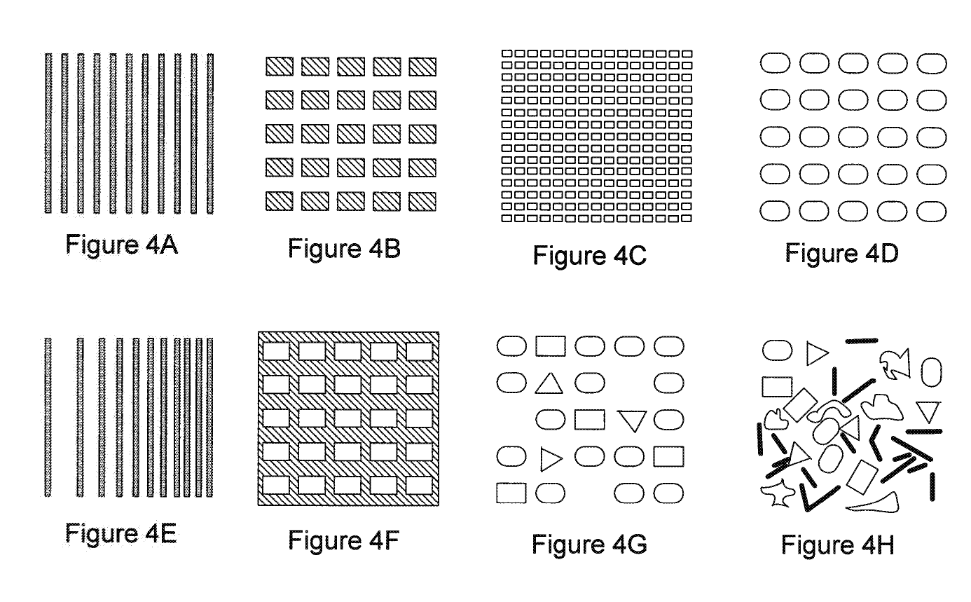

FIGS. 4A-4H depicts examples of different types of ferromagnetic MFG structures that may be employed with magnetic trapping stations this invention. Eight different ferromagnetic element patterns are shown in the figure. These are employed to shape a magnetic field gradient originating from an external source of a magnetic field (not shown). As shown, the ferromagnetic structures are provided in an organized pattern, such as parallel lines, an orthogonal grid, and rectangular arrays of regular or irregular geometric shapes. The structures may be regular or reticulated as shown. Other embodiments, not shown, may employ parallel stripes, etc.

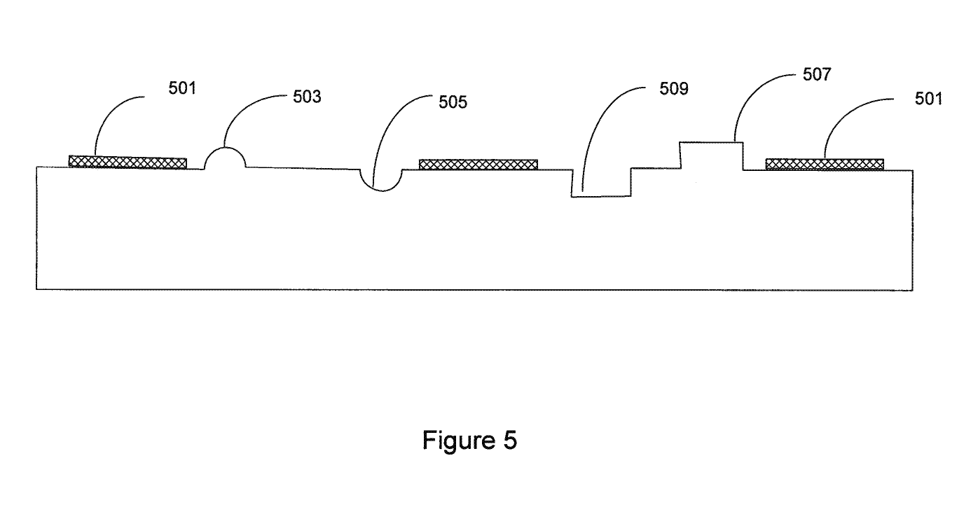

Generally, the features or elements in these patterns may be made from various materials having permeabilities that are significantly different from that of the fluid medium in the device (e.g., the buffer). As indicated, the elements may be made from a ferromagnetic material. In a specific embodiment, the patterns are defined by nickel features on a glass or polymer substrate. In alternative embodiments, the MFG structures are combined with other types of capture structures such as electrodes, specific binding moieties (e.g., regions of nucleotide probes or antibodies), physical protrusions or indentations, etc. FIG. 5 presents examples of non-magnetic capture features that are fabricated among a soft-magnetic (e.g., nickel) pattern. The patterns may be positive (503 and 507) or negative (505 and 509) surface features to facilitate laminar mixing of the fluid over the nickel structures (501), causing enhanced magnetic trapping.

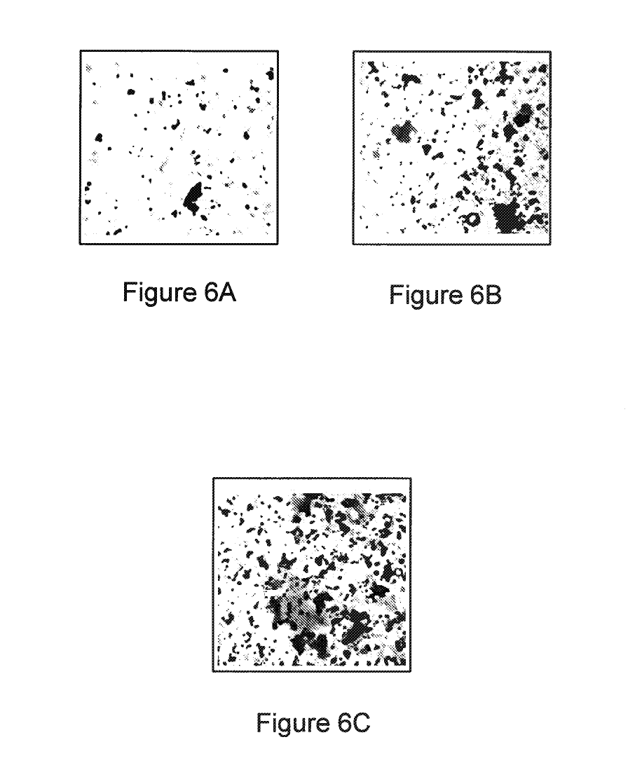

Other types of MFG structures comprise ferromagnetic materials that do not form well-defined shapes or regular features. Instead, the structures form randomly placed features such as randomly dispersed powder, filings, granules, etc. These structures are affixed to one or more walls of the trapping station adhesives, pressure bonding, etc. FIG. 6 shows examples of random array of ferromagnetic structures from left to right: 5%, 10% and 30% nickel powder in an epoxy resin. Such structures have found to be effective MFG elements in magnetic trapping stations.

In an alternative embodiment, the trapping station contains no MFG structures. Instead, magnetic capture is based solely on the strength of the external magnetic field, without the aid of a field shaping element such as MFG structures.

Fluidics and Sorting Chamber Design

While some embodiments of this invention are implemented in micro-scale microfluidic systems, it should be understood that methods, apparatus, and systems of this invention are not limited to microfluidic systems. Typical sizes of larger trapping chambers range between about 1 and 100 millimeters in length (in the direction of flow), between about 1 and 100 millimeters in width and between about 1 micrometer and 10 millimeters depth (although typically about 1 millimeter or less). The depth and width together define the cross section through which fluid flows. The depth represents the dimension in the direction that the magnetic field penetrates into the channel, typically a direction pointed away from the position of the external magnet. In certain embodiments, the chambers have an aspect ratio (length to width) that is greater than 1, e.g., about 2 to 8.

In general, the applied magnetic field should be sufficiently great to capture or trap magnetic particles flowing in a fluid medium. Those of skill in the art will recognize that the applied magnetic force must be significantly greater than the hydrodynamic force exerted on the particles by the flowing fluid. This may limit the depth dimension of the trapping station.

In certain embodiments, the integrated fluidics systems are microfluidic systems. Microfluidic systems may be characterized by devices that have at least one “micro” channel. Such channels may have at least one cross-sectional dimension on the order of a millimeter or smaller (e.g., less than or equal to about 1 millimeter). Obviously for certain applications, this dimension may be adjusted; in some embodiments the at least one cross-sectional dimension is about 500 micrometers or less. In some embodiments, as applications permit, a cross-sectional dimension is about 100 micrometers or less (or even about 10 micrometers or less—sometimes even about 1 micrometer or less). A cross-sectional dimension is one that is generally perpendicular to the direction of centerline flow, although it should be understood that when encountering flow through elbows or other features that tend to change flow direction, the cross-sectional dimension in play need not be strictly perpendicular to flow. Often a micro-channel will have two or more cross-sectional dimensions such as the height and width of a rectangular cross-section or the major and minor axes of an elliptical cross-section. Either of these dimensions may be compared against sizes presented here. Note that micro-channels employed in this invention may have two dimensions that are grossly disproportionate—e.g., a rectangular cross-section having a height of about 100-200 micrometers and a width on the order or a centimeter or more. Of course, certain devices may employ channels in which the two or more axes are very similar or even identical in size (e.g., channels having a square or circular cross-section).

Often a controller will be employed to coordinate the operations of the various systems or sub-systems employed in the overall microfluidic system. Such controller will be designed or configured to direct the sample through a microfluidic flow passage. It may also control other features and actions of the system such as the strength and orientation of a magnetic field applied to fluid flowing through the microfluidic device, control of fluid flow conditions within the microfluidic device by actuating valves and other flow control mechanisms, mixing of magnetic particles and sample components in an attachment system, generating the sample (e.g., a library in a library generation system), and directing fluids from one system or device to another. The controller may include one or more processors and operate under the control of software and/or hardware instructions.

Integration

Examples of operational modules that may be integrated with magnetic trapping sorters in fluidics devices include (a) additional enrichment modules such as fluorescence activated cell sorters and washing modules, (b) reaction modules such as sample amplification (e.g., PCR) modules, restriction enzyme reaction modules, nucleic acid sequencing modules, target labeling modules, chromatin immunoprecipitation modules, crosslinking modules, and even cell culture modules, (c) detection modules such as microarrays of nucleic acids, antibodies or other highly specific binding agents, and fluorescent molecular recognition modules, and (d) lysis modules for lysing cells, disrupting viral protein coats, or otherwise releasing components of small living systems. Each of these modules may be provided before or after the magnetic sorter. There may be multiple identical or different types of operational modules integrated with a magnetic sorter in a single fluidics system. Further, one or more magnetic sorters may be arranged in parallel or series with respect to various other operational modules. Some of these operational modules may be designed or configured as traps in which target species in a sample are held stationary or generally constrained in particular volume.

As should be apparent from the above examples of modules, operations that may be performed on target and/or non-target species in modules of integrated fluidics devices include sorting, coupling to magnetic particles (sometimes referred to herein as “labeling”), binding, washing, trapping, amplifying, removing unwanted species, precipitating, cleaving, diluting, ligating, sequencing, synthesis, labeling (e.g., staining cells), cross-linking, culturing, detecting, imaging, quantifying, lysing, etc.

Specific examples of biochemical operations that may be performed in the magnetic sorting modules of integrated fluidic devices include synthesis, purification, and/or screening of plasmids, aptamers, proteins, and peptides; evaluating enzyme activity; and derivatizing proteins and carbohydrates. A broad spectrum of biochemical and electrophysiological assays may also be performed, including: (1) genomic analysis (sequencing, hybridization), PCR and/or other detection and amplification schemes for DNA, and RNA oligomers; (2) gene expression; (3) enzymatic activity assays; (4) receptor binding assays; and (5) ELISA assays. The foregoing assays may be performed in a variety of formats, such as: homogeneous, bead-based, and surface bound formats. Furthermore, devices as described herein may be utilized to perform continuous production of biomolecules using specified enzymes or catalysts, and production and delivery of biomolecules or molecules active in biological systems such as a therapeutic agents. Microfluidic devices as described herein may also be used to perform combinatorial syntheses of peptides, proteins, and DNA and RNA oligomers as currently performed in macrofluidic volumes.

One increasingly important example operation using the apparatuses and methods of the present invention is automated protein purification, particularly as protein is expressed in cell culture. Protein purification may be performed manually. However, the apparatuses and methods of the present invention provide a time and labor saving automation that delivers a high purity product with low cost.

In a prophetic example, desired proteins are expressed in organisms such as virus, bacteria, insect or mammalian cells. The expressed protein may be designed such that it may be selectively isolated from background materials. This may be accomplished via adding one or more selectable amino acid tags that add a stretch of amino acid to the protein. The tag may be a His tag, FLAG tag or other epitope-based tags (E-tags). The cells (for example) are introduced to one of the sample reservoirs described herein, with magnetic particles and lyses reagents in the same or one or more reservoirs. The magnetic particles may be magnetic beads coated with a high affinity media such as NTA-agarose or other resin containing to nickel. Mixing between the various sample reservoirs is promoted via one or more of the techniques described above, e.g., pneumatic, hydraulic, or magnetic mixing. The cells are disrupted by the lysing reagent and, under suitable conditions, the magnetic particles bind with the target protein in the lysate. The raw lysate is then flowed into the magnetic separation chamber where the beads become trapped on the surface of the channel. Wash buffer is added to elute the untagged and unbound protein and other cell fragments. According to various embodiments, the magnetic separation chamber may be agitated magnetically or through other means to further remove any unbound protein stuck between trapped particles. A highly stringent wash buffer may be used to further elute unwanted particles. At this point, only the target protein and bound magnetic particles remain in the chamber with very high selectivity. The target protein may be released by using a bead release agent into a small volume, optionally for further processing. Lastly, the magnetic particles may be released. Because these various operations occur on a unitary or disposable cartridge in a machine, the procedure may be preprogrammed and automated to save time and cost. This configuration may be used to selectively trap other nucleic acid related products, such as RNA, which may be so labeled so as to be similarly selectable.

The present fluidic sorting devices may be integrated such that they are configured for particular purposes. For example, one may desire to have one mixing reservoir and several trapping stations. In this way, a single sample is deployed and mixed with magnetic particles (for example), such that selected targets are labeled. This single sample is routed to greater than one, or a plurality of trapping stations. The trapping stations may be configured in parallel or in series. Optionally, one or more aspects of this parallel-trapping station configuration may be under the control of a single controller for mixing, disposing on the trapping station, and eluting (such that the labeled target species are maintained in the trapping station, for example). When connected in series, target species concentration may be improved by sequential trapping to remove any incidental non-specific binding. In addition, a series trapping configuration may be used when two or more markers are required to for certain target cells, such as tumor cells. In that case, one trapping station may isolate cells having one marker (such as a first cell surface receptor) and then the selected cells may be washed so as to remove the magnetic particle (for example). The population of selected cells may be then mixed with markers for another target, such as a second cell surface receptor. The cells so labeled for this second cell surface receptor may then be trapped. After eluting (for example) the non-trapped cells, the final population will be those cells that display both the first and second cell surface receptors. This process may be repeated to collect further subpopulations. Alternatively, one may desire to remove certain targets, such as subpopulations having a first receptor but not a second cell surface receptor, for example. This process may be repeated, and the present devices may be configured, to facilitate a variety of multiple-target trapping iterations Analogous methods and device configurations may be used for selecting subpopulations of a variety of target molecules in a sample including but not limited to cell surface receptors, molecular moieties, or other types of selectable targets

Examples of Reactors and Lysis Modules in Fluidics Systems

Various features may be employed in a microfluidic reactor employed in an integrated device of this invention. The exact design and configuration will depend on the type of reaction: thermal management system, micromixers, catalyst structures and a sensing system. In certain embodiments, a thermal management system includes heaters, temperature sensors and heat transfer (micro heat exchanges). In microreactors, all components can be integrated in resulting in a very precise control of temperatures which is crucial for instance in PCR for DNA amplification.

Micromixers may be used for mixing two solutions (e.g. a sample and a reagent) to make the reaction possible. In microscale systems, mixing often relies on diffusion due to the laminar behavior of fluid at low Reynolds numbers. In one embodiment, a hydrophobic material defining a hole separates two adjacent chambers. When aqueous solutions are used, the hydrophobicity of the interface permits both chambers to be filled with fluid plugs without mixing. A pressure gradient can then be applied to force fluid through the hole in the hydrophobic layer to induce diffusion between the two plugs. In one embodiment, the hole is actually a slit in which no material is removed from the intermediate dividing layer.

Catalyst structures may be employed to accelerate a chemical reaction (e.g., cross-linking or sequencing). In microreactors, the catalyst can be implemented in the form of, e.g., fixed beads, wires, thin films or a porous surface. While beads and wires and not compatible with batch fabrication, thin films and porous surface catalysts can be integrated in the fabrication of microreactors.

A sensing system may employ chemical microsensors or biosensors, for example. Designing a microreactor with glass or plastic provides optical access to the reaction chamber and thus, all optical measurement methods.

Before the contents of a biological cell may be analyzed, the cells to be analyzed are made to burst so that the components of the cell can be separated. The methods of cell disruption used to release the biological molecules in a cell and in a virus include, e.g., electric field, enzyme, sonication, and using a detergent. Mechanical forces may also be used to shear and burst cell walls.

Cell lysis may be performed by subjecting the cells trapped in a reaction chamber to pulses of high electric field strength, typically in the range of about 1 kV/cm to 10 kV/cm. The use of enzymatic methods to remove cell walls is well-established for preparing cells for disruption, or for preparation of protoplasts (cells without cell walls, as in plant cells, for example) for other uses such as introducing cloned DNA or subcellular organelle isolation. The enzymes are generally commercially available and, in most cases, were originally isolated from biological sources (e.g. snail gut for yeast or lysozyme from hen egg white). The enzymes commonly used include lysozyme, lysostaphin, zymolase, cellulase, mutanolysin, glycanases, proteases, mannase etc. In accordance with various embodiments, the cell lysis enzyme may be added to the trapping chamber from a separate reservoir or be mixed with the sample in the beginning.

In addition to potential problems with the enzyme stability, the susceptibility of the cells to the enzyme can be dependent on the state of the cells. For example, yeast cells grown to maximum density (stationary phase) possess cell walls that are notoriously difficult to remove whereas midlog growth phase cells are much more susceptible to enzymatic removal of the cell wall. If an enzyme is used, it may have to be sorted and removed from the desired material before further analysis.

Sonication uses a high-frequency wave that mechanically burse the cell walls. Ultrasound at typically 20-50 kHz is applied to the sample via a metal probe that oscillates with high frequency. The probe is placed into the cell-containing sample and the high-frequency oscillation causes a localized high pressure region resulting in cavitation and impaction, ultimately breaking open the cells. Cell disruption is available in smaller samples (including multiple samples under 200 μL in microplate wells) and with an increased ability to control ultrasonication parameters. The present invention may be used with a thermal management system as described above such that the sample is kept in cool conditions, for example, to avoid undue heat due to sonication, where the heat may denature the desired protein.

Detergent-based cell lysis is an alternative to physical disruption of cell membranes, although it is sometimes used in conjunction with homogenization and mechanical grinding. Detergents disrupt the lipid barrier surrounding cells by disrupting lipid:lipid, lipid:protein and protein:protein interactions. The ideal detergent for cell lysis depends on cell type and source and on the downstream applications following cell lysis. Animal cells, bacteria and yeast all have differing requirements for optimal lysis due to the presence or absence of a cell wall. Because of the dense and complex nature of animal tissues, they require both detergent and mechanical lysis to effectively lyse cells.

In general, nonionic and zwitterionic detergents are milder, resulting in less protein denaturation upon cell lysis, than ionic detergents and are used to disrupt cells when it is critical to maintain protein function or interactions. CHAPS, a zwitterionic detergent, and the Triton X series of nonionic detergents are commonly used for these purposes. In contrast, ionic detergents are strong solubilizing agents and tend to denature proteins, thereby destroying protein activity and function. SDS, and ionic detergent that binds to and denatures proteins, is used extensively for studies assessing protein levels by gel electrophoresis and western blotting. If protein purification is desired, and the cells have partitioned the protein into sub-cellular membrane bound moieties, such as inclusion bodies, other detergents, such as the commercially available TWEEN may be used as an additional reagent to disrupt such inclusion bodies.

A mechanical method for cell disruption uses glass or ceramic beads and a high level of agitation to shear and burst cell walls. This process works for easily disrupted cells, is inexpensive, but has integration issues for the micorfluidic device. In one embodiment, beads are used in a closed chamber holding the sample and are agitated with an electric motor. In other embodiments, high pressure is applied to fluid containing the cell samples while forcing the fluid to flow through a very narrow channel. Shear between the cell and channel walls under such conditions would disrupt the cell.

Examples of Detectors in Integrated Flow Systems

In various applications envisaged for integrated microsystems it will be necessary to quantify the material present in a channel at one or more positions similar to conventional laboratory measurement processes. Techniques typically utilized for quantification include, but are not limited to, optical absorbance, refractive index changes, fluorescence emission, chemiluminescence, various forms of Raman spectroscopy, electrical conductometric measurements, impedance measurements (e.g., impedance cytometry) electrochemical amperiometric measurements, acoustic wave propagation measurements.

Optical absorbance measurements are commonly employed with conventional laboratory analysis systems because of the generality of the phenomenon in the UV portion of the electromagnetic spectrum. Optical absorbance is commonly determined by measuring the attenuation of impinging optical power as it passes through a known length of material to be quantified. Alternative approaches are possible with laser technology including photo acoustic and photo thermal techniques. Such measurements can be utilized with the integrated fluidics devices discussed here with the additional advantage of potentially integrating optical wave guides on microfabricated devices. The use of solid-state optical sources such as LEDs and diode lasers with and without frequency conversion elements would be attractive for reduction of system size.

Refractive index detectors have also been commonly used for quantification of flowing stream chemical analysis systems because of generality of the phenomenon but have typically been less sensitive than optical absorption. Laser based implementations of refractive index detection could provide adequate sensitivity in some situations and have advantages of simplicity. Fluorescence emission (or fluorescence detection) is an extremely sensitive detection technique and is commonly employed for the analysis of biological materials. This approach to detection has much relevance to miniature chemical analysis and synthesis devices because of the sensitivity of the technique and the small volumes that can be manipulated and analyzed (volumes in the picoliter range are feasible). For example, a 100 pL sample volume with 1 nM concentration of analyte would have only 60,000 analyte molecules to be processed and detected. There are several demonstrations in the literature of detecting a single molecule in solution by fluorescence detection. A laser source is often used as the excitation source for ultrasensitive measurements but conventional light sources such as rare gas discharge lamps and light emitting diodes (LEDs) are also used. The fluorescence emission can be detected by a photomultiplier tube, photodiode or other light sensor. An array detector such as a charge coupled device (CCD) detector can be used to image an analyte spatial distribution.

Raman spectroscopy can be used as a detection method for microfluidic devices with the advantage of gaining molecular vibrational information, but with the disadvantage of relatively poor sensitivity. Sensitivity has been increased through surface enhanced Raman spectroscopy (SERS) effects but only at the research level. Electrical or electrochemical detection approaches are also of particular interest for implementation on microfluidic devices due to the ease of integration onto a microfabricated structure and the potentially high sensitivity that can be attained. The most general approach to electrical quantification is a conductometric measurement, i.e., a measurement of the conductivity of an ionic sample. The presence of an ionized analyte can correspondingly increase the conductivity of a fluid and thus allow quantification. Amperiometric measurements imply the measurement of the current through an electrode at a given electrical potential due to the reduction or oxidation of a molecule at the electrode. Some selectivity can be obtained by controlling the potential of the electrode but it is minimal. Amperiometric detection is a less general technique than conductivity because not all molecules can be reduced or oxidized within the limited potentials that can be used with common solvents. Sensitivities in the 1 nM range have been demonstrated in small volumes (10 nL). The other advantage of this technique is that the number of electrons measured (through the current) is equal to the number of molecules present. The electrodes required for either of these detection methods can be included on a microfabricated device through a photolithographic patterning and metal deposition process. Electrodes could also be used to initiate a chemiluminescence detection process, i.e., an excited state molecule is generated via an odixation-reduction process which then transfers its energy to an analyte molecule, subsequently emitting a photon that is detected.

Acoustic measurements can also be used for quantification of materials but have not been widely used to date. One method that has been used primarily for gas phase detection is the attenuation or phase shift of a surface acoustic wave (SAW). Adsorption of material to the surface of a substrate where a SAW is propagating affects the propagation characteristics and allows a concentration determination. Selective sorbents on the surface of the SAW device are often used. Similar techniques may be useful in the devices described herein.

The mixing capabilities of the microfluidic systems lend themselves to detection processes that include the addition of one or more reagents. Derivatization reactions are commonly used in biochemical assays. For example, amino acids, peptides and proteins are commonly labeled with dansylating reagents or o-phthaldialdehyde to produce fluorescent molecules that are easily detectable. Alternatively, an enzyme could be used as a labeling molecule and reagents, including substrate, could be added to provide an enzyme amplified detection scheme, i.e., the enzyme produces a detectable product. There are many examples where such an approach has been used in conventional laboratory procedures to enhance detection, either by absorbance or fluorescence. A third example of a detection method that could benefit from integrated mixing methods is chemiluminescence detection. In these types of detection scenarios, a reagent and a catalyst are mixed with an appropriate target molecule to produce an excited state molecule that emits a detectable photon.

What is claimed is:

1. A fluidic sorting device comprising:
(a) one or more reservoirs on the fluidics device designed to receive a sample and magnetic particles in a fluid medium;
(b) a mechanism for mixing the sample and magnetic particles in the fluid medium to label one or more species in the sample with said magnetic particles;
(c) a fluidic sorting chamber having (i) an inlet for receiving labeled sample in the fluid medium, (ii) an outlet for allowing the fluid medium to exit the fluidic chamber, and (iii) a surface for retaining the magnetic particles captured by a magnetic field; and
(d) an external source of the magnetic field in the fluidic sorting chamber.

2. The device of claim 1, wherein the fluidics device is a unitary device.

3. The device of claim 1, wherein the fluidics device is a disposable device.

4. The device of claim 1, wherein the one or more reservoirs is designed to further receive a selection entity.

5. The device of claim 1, comprising two reservoirs, one for receiving the sample and the other for receiving the magnetic particles.

6. The device of claim 1, wherein the mechanism for mixing the sample and the functionalized magnetic particles comprises a pneumatic mixing system.

7. The device of claim 6, wherein the pneumatic mixing system is designed or adapted to alternatively apply pneumatic pressure to two reservoirs, one for receiving the sample and the other for receiving the magnetic particles, to thereby facilitate labeling of a species in the sample.

8. The device of claim 1, further comprising a source of functionalized magnetic particles comprising a functional agent for specifically binding to a species in the sample.

9. The device of claim 1, wherein the external source of the magnetic field comprises a single permanent magnet.

10. The device of claim 1, wherein the external source of the magnetic field comprises a plurality of permanent magnets.

11. The device of claim 1, wherein the fluidic sorting chamber further comprises a magnetic field gradient generator for exerting a magnetic force on a sample to capture, at least temporarily, magnetic particles in the fluid medium.

12. The device of claim 1, wherein the fluidic sorting chamber has at least one sub-millimeter dimension.

13. The device of claim 10, further comprising a mechanism for moving the external source of the magnetic field by inserting the individual magnets of the plurality of magnets sequentially with respect to the surface for retaining magnetic particles.

14. A method for labeling and trapping a species in a sample at a trapping station of a fluidics device that includes (i) one or more reservoirs on the fluidics device designed or adapted to receive a sample and magnetic particles in a fluid medium, (ii) one or more fluidic sorting chambers, and (iii) an external source of a magnetic field in the fluidic sorting chamber, the method comprising:
(a) adding the sample and the magnetic particles to the one or more reservoirs;
(b) mixing the sample and magnetic particles in the fluid medium to label one or more species in the sample with said magnetic particles;
(c) flowing the labeled sample into the one or more fluid sorting chambers; and
(d) trapping magnetic particles on a surface of the fluidic chamber.

15. The method of claim 14, wherein trapping the magnetic particles comprises moving the external source of the magnetic field with respect to the fluidic sorting chamber while the magnetic particles flow through the fluidics device in the fluid medium to trap magnetic particles in a substantially uniform fashion on a surface of the fluidic chamber.

16. The method of claim 14, further comprising flowing a release reagent to the fluidic chamber to release bound species in the sample from the magnetic particles and collecting the one or more species in the sample.

17. The method of claim 14, wherein the flowing operation occurs simultaneously into the more than one fluid sorting chambers.

18. A method of claim 14, wherein the sample is a nucleic acid expression product, said product selected from a group consisting of protein and RNA.

19. A method of labeling and trapping a protein species from a cell lysate at a trapping station of a fluidics device that includes (i) one or more reservoirs on the fluidics device designed or adapted to hold a cell lysate sample containing the target protein species and magnetic particles in a fluid medium, (ii) one or more fluidic sorting chambers, and (iii) an external source of a magnetic field in the fluidic sorting chamber, the method comprising:
(a) providing the cell lysate sample and the magnetic particles to the one or more reservoirs;
(b) mixing the cell lysate and magnetic particles in the fluid medium under conditions suitable to label one or more protein species with said magnetic particles;
(c) flowing the labeled cell lysate into the one or more fluid sorting chambers; and
(d) trapping magnetic target protein species on a surface of the fluidic chamber.

20. A method of claim 19, wherein the providing the cell lysate comprises lysing cells in-situ in the one or more reservoirs.

21. A method of claim 19, wherein the protein encodes one or more detectable amino acid tags.

22. A fluidic sorting device comprising:
(a) a fluidic sorting chamber having (i) one or more inlets for receiving a fluid medium, (ii) one or more outlets for allowing the fluid medium to exit the fluidic sorting chamber, (iii) a surface for retaining the magnetic particles captured by a magnetic field, and (iv) one or more valves to constrain the fluid medium to the fluidic sorting chamber;
(b) an external source of the magnetic field in the fluidic sorting chamber; and
(c) a mechanism for varying the magnetic field produced by the external source of the magnetic field within the fluid sorting chamber after trapping to move the magnetic particles in the sorting chamber.

23. The fluidic sorting device of claim 22, further comprising a source of reagent for releasing bound components from said magnetic particles, wherein said source of reagent is coupled to said fluidic sorting chamber.

24. The fluidic sorting device of claim 22, wherein the one or more valves are disposed upstream and downstream of the fluidic sorting chamber.

25. The fluidic sorting device of claim 22, wherein the external source of the magnetic field comprises a plurality of permanent magnets arranged in an array.

26. The fluidic sorting device of claim 22, wherein the external source of the magnetic field comprises two magnets or two pluralities of permanent magnets located on opposing sides of the fluidic sorting chamber.

27. The fluidic sorting device of claim 26, wherein the mechanism for varying the magnetic field produced by the external source of the magnetic field comprises a feature for moving the two magnets or two pluralities of permanent magnets toward and away from the fluidic sorting chamber.

28. A method for trapping and releasing species in a sample at a trapping station of a fluidics device that includes (i) a fluidic sorting chamber and (ii) an external source of the magnetic field in the fluidic sorting chamber, the method comprising:
(a) flowing a sample comprising some components labeled with magnetic particles into the fluid sorting chamber;
(b) trapping magnetic particles and associated sample components on a surface of the fluidic chamber;
(c) contacting the trapped magnetic particles and sample components with a release agent; and
(d) causing the magnetic particles and associated sample components to move about within a fluid medium in the sorting chamber to thereby facilitate release of the sample components from the magnetic particles.

29. The method of claim 28, where causing the magnetic particles and associated sample components to move about within the fluid medium comprises varying a magnetic field applied to the sorting chamber.

30. A fluidic sorting device comprising one or more reservoirs for combining a moiety within a fluid sample with a magnetic particle and a pneumatic mechanism for mixing the sample with magnetic particles.

31. A fluidic sorting device of claim 30 further comprising a fluidic sorting chamber having (i) an inlet for receiving labeled sample, (ii) an outlet for allowing the fluid to exit the fluidic chamber, and (iii) a surface for retaining the magnetic particles captured by a magnetic field; and, an external source of the magnetic field in the fluidic sorting chamber.
